(19) United States
(12) Patent Application Publication
(10) Pub. No.: US 2022/0275369 A1
(43) Pub. Date: Sep. 1, 2022

(54) ANTISENSE THERAPY FOR PTP1B RELATED CONDITIONS

(71) Applicant: MURDOCH UNIVERSITY, Murdoch (AU)

(72) Inventors: Rakesh Veedu, Bullcreek (AU); Suxiang Chen, Murdoch (AU)

(21) Appl. No.: 17/286,017

(22) PCT Filed: Sep. 18, 2019
(86) PCT No.: PCT/AU2019/050996

§ 371 Date: Apr. 16, 2021

(30) Foreign Application Priority Data
Oct. 18, 2018 (AU) 2018903950

(51) Int. Cl. C12N 15/113; A61P 35/00

(52) U.S. Cl. CPC C12N 15/1137; A61P 35/00; C12N 2310/11

(57) ABSTRACT

An isolated or purified antisense oligomer targeted to a nucleic acid molecule encoding PTPN1 pre-mRNA, wherein the antisense oligomer inhibits the expression of PTP1B.

### CROSS-REFERENCE TO RELATED APPLICATIONS

This application is a U.S. National Phase of International Application No. PCT/AU2019/050996 filed Sep. 18, 2019, which claims priority to Australian Patent Application No. 2018903950 filed Oct. 18, 2018.

### INCORPORATION BY REFERENCE OF MATERIALS SUBMITTED ELECTRONICALLY

The Sequence Listing, which is a part of the present disclosure, is submitted concurrently with the specification as a text file. The name of the text file containing the Sequence Listing is “56629_Seqlisting.txt”, which was created on Nov. 8, 2021 and is 7,125 bytes in size. The subject matter of the Sequence Listing is incorporated herein in its entirety by reference.

### TECHNICAL FIELD

The present invention relates to antisense oligomers (ASO) to facilitate modification of isoform production in the protein tyrosine phosphatase-1B (PTP1B) protein coded by gene PTPN1. The invention further provides methods to treat, prevent or ameliorate the effects of insulin resistance, leptin resistance by administration of antisense oligomers (ASO) and therapeutic compositions comprising antisense oligomers to the PTPN1 gene.

### BACKGROUND ART

The following discussion of the background art is intended to facilitate an understanding of the present invention only. The discussion is not an acknowledgement or admission that any of the material referred to is or was part of the common general knowledge as at the priority date of the application.

Type 2 diabetes (T2DM) is a metabolic disorder characterized by insufficient secretion or inefficient processing of insulin, a hormone secreted by pancreatic beta cells which plays a central role in glucose metabolism. Defects of insulin results in increased glucose concentration in plasma, while the state of high glucose persistently exists, leads to hyperglycemia that eventually progresses to T2DM. Insulin defects stem from upstream and downstream dysregulations that are referred to as pancreatic beta cell dysfunction (upstream) and insulin resistance (downstream), respectively. Beta cell dysfunction results in a reduction in insulin production while insulin resistance is described as interruptions occurring during insulin signal transduction pathway in glucose recipient cells. Upstream and downstream insulin dysregulations interact with each other with a complex interrelationship and collectively contribute to the pathogenesis of T2DM. To summarize, beta cell dysfunction and insulin resistance are two root causes of T2DM.

Obesity is a key accelerator of Type 2 diabetes (T2DM) and is characterized by resistance of leptin. Leptin is an adipocyte-derived hormone which acts on the hypothalamus to decrease food intake and increase energy expenditure. Obesity is associated with interruptions occurring during leptin signal transduction pathway (leptin resistance) leading to increased food intake and reduced energy expenditure.

Expression of PTP1B negatively regulates both insulin signalling and leptin signalling pathways, thus PTP1B is a therapeutic target for both T2DM and obesity.

Normal therapy for T2DM is based on various oral hypoglycaemic agents, which are currently prescribed antidiabetic drug therapies which universally aims to reduce blood glucose levels by targeting one or more of the six key organs and/or tissues, i.e., the pancreas, liver, skeletal muscle, small intestine, kidneys, and adipose tissue. To date, none of the pharmacological agents have shown promise in halting the underlying causes of T2DM, namely, pancreatic beta cell dysfunction and insulin resistance.

PTP1B plays a role as an oncogene. The PTP1B gene is frequently amplified in ovarian, gastric, prostate and breast cancers, and correlates with poor prognosis. Knockdown of PTP1B reduces cell growth, induces both cell cycle arrest and apoptosis, and reduces cancer cell migration and invasion by reversing the epithelial—mesenchymal transition (EMT) process. Thus, PTP1B is also a therapeutic target for solid cancers.

The present invention seeks to provide a composition and method to reduce the effects of insulin resistance, T2DM, leptin resistance, obesity, and solid cancers or to provide the consumer with a useful or commercial choice.

### SUMMARY OF INVENTION

The present invention is based on the surprising discovery that the use of isolated or purified antisense oligomers (ASO) for modifying pre-mRNA splicing production of PTPN1 to increase the production of truncated, nonsense or prematurely terminated proteins, such as proteins with pre-mature stop codons, may lead to a reduction in production of functional PTP1B protein.

Broadly, according to one aspect of the invention, there is provided an isolated or purified antisense oligomer (ASO) for modifying pre-mRNA splicing in the protein tyrosine phosphatase-1B (PTP1B) protein coded by the PTPN1 gene transcript or part thereof. Preferably, there is provided an isolated or purified antisense oligomer for inducing splice modulation, especially exon skipping leading to pre-mature termination codons that results in reduced production of the full length PTPN1 gene transcript or part thereof.

Preferably, the antisense oligomer is a phosphorodiamidate morpholino oligomer.

Preferably, the antisense oligomer is selected from the group comprising the sequences set forth in Table 1. Preferably, the antisense oligomer is selected from the list comprising: SEQ ID NO: 1-4, 10-15, 18, 19, 23-25, 27, 29, 31-41. Preferably, the antisense oligomer used in the present invention is chosen from the list comprising: SEQ ID NO: 1, or 32-36. More preferably, the antisense oligomer used in the present invention is SEQ ID NO: 33.

The invention extends, according to a still further aspect thereof, to cDNA or cloned copies of the antisense oligomer sequences of the invention, as well as to vectors containing the antisense oligomer sequences of the invention. The invention extends further also to cells containing such sequences and/or vectors.

There is also provided a method for manipulating splicing factor binding in a PTPN1 gene transcript, the method including the step of:
providing one or more of the antisense oligomers as described herein and allowing the oligomer(s) to bind to a target nucleic acid site.

There is also provided a pharmaceutical, prophylactic, or therapeutic composition to treat, prevent or ameliorate the effects of a disease associated with the PTP1B protein in a subject, the composition comprising:
one or more antisense oligomers as described herein and
one or more pharmaceutically acceptable carriers and/or diluents.

Preferably, the disease conditions associated with the PTP1B protein is insulin resistance, Type 2 diabetes (T2DM), leptin resistance, obesity, and solid cancers.

The subject with the disease associated with the PTP1B protein may be a mammal, including a human.

There is also provided a method to treat, prevent or ameliorate the effects of a disease associated with the PTP1B protein, comprising the step of:
administering to the subject an effective amount of one or more antisense oligomers or pharmaceutical composition comprising one or more antisense oligomers as described herein.

There is also provided the use of purified and isolated antisense oligomers as described herein, for the manufacture of a medicament to treat, prevent or ameliorate the effects of a disease associated with the PTP1B protein.

There is also provided a kit to treat, prevent or ameliorate the effects of a disease associated with the PTP1B protein in a subject, which kit comprises at least an antisense oligomer as described herein and combinations or cocktails thereof, packaged in a suitable container, together with instructions for its use.

Further aspects of the invention will now be described with reference to the accompanying non-limiting Examples and Drawings.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is an exon map of the human PTPN1-201 transcript, and mouse Ptpn1-201 transcript.

FIG. 2 is a Northern blot showing the transfection efficiency of AO 1-8 in Huh-7 cell line at 400 nanomolar concentration. S: scrambled sequence, UT: untreated, NC: negative control, Imax: RNAiMAX, pro: Metafectene® PRO, si: Metafectene® SI⁺.

FIG. 3 is a Northern blot showing the transfection efficiency of AO 9-16 using RNAiMAX reagent in Huh-7 cell line at 400 nanomolar concentration. S: scrambled sequence, UT: untreated, NC: negative control.

FIG. 4 is a Northern blot showing the transfection efficiency of AO 17-30 using L3K reagent in Huh-7 cell line at 400 nanomolar concentration. S: scrambled sequence, UT: untreated, NC: negative control.

FIG. 5 is a Northern blot showing the dose response test of AO 1 and AO 4. Concentrations include 400, 200, 100, 50, 25, 12.5 nanomolar in Huh-7 cell line. S: scrambled sequence, UT: untreated, NC: negative control.

FIG. 6 is a schematic illustration of modified transfection protocols used for HepG2 transfection experiments based on RNAiMAX and Lipofectamine 3000 (L3K) manufacturer's instructions. 2′OMePS form of AO 1 was used in this experiment. The stock concentration of AO 1 is 181171 nanomolar. Opti is short for Opti-MEM™ I reduced serum media.

FIG. 7 is a Northern blot showing the different transfection reagents (RNAiMAX and L3K) and different transfection protocols (RNAiMAX: 1.1-1.7; L3K: 2.1 and 2.2) have been tested in HepG2 cell line and results showed that protocol 1.3 (reverse transfection protocol of RNAiMAX) is the best protocol for 2′OMePS antisense oligonucleotide transfection to HepG2. 2′OMePS form of AO 1 was used in this experiment.

FIG. 8 is a Northern blot showing comparison of exon-2 skipping efficiency between PTPN1 1E2A (+1+25) (AO1), 2′-OMePS form of ISIS 107773, PTPN1 1E2A (+1+23) (AO 31), and PTPN1 1 E2A (+3+27) (AO 32) at the concentration of 400 nanomolar in HepG2.

FIG. 9 is a representation of Sanger sequencing result confirmed that AO1, PTPN1 1E2A (+1+25) induces exon-2 skipping during the transcription process of the gene PTPN1.

FIG. 10 is a Northern blot showing comparison of exon-2 skipping efficiency and non-skipping product knockdown efficiency between PTPN1 1 E2A (+1+25) (AO1), PTPN1 1 E2A (+3+27) (AO 32), 2′-OMePS form of ISIS 107773, and ISIS 107773 (5-10-5 MOE gapmer) at the concentration of 400 nanomolar in triplicates in HepG2.

FIG. 11 is a Northern blot showing the dose response test of PTPN1 1E2A (+1+25) (AO 1). Concentrations include 400, 200, 100, 50, 25, 12.5, 6.3, 3.1 nanomolar in HepG2.

FIG. 12 is a Northern blot showing the transfection efficiency of AO 1, 32-36 using RNAiMAX reagent in IHH cell line at 400 nanomolar concentration.

FIG. 13 is a Northern blot showing comparison of exon-2 skipping efficiency and non-skipping product knockdown efficiency between AO 1, AO 32-36, 2′-OMePS form of ISIS 107773, and ISIS 107773 (5-10-5 MOE gapmer) at the concentration of 400 nanomolar using RNAiMAX reagent in HepG2, Huh-7, and IHH cell lines.

FIG. 14 is a Northern blot showing the dose response test of 2′OMePS form of PTPN1 1E2A (+5+29) (AO 33) (Diabexa-2) in HepG2 and IHH cells. Concentrations include 400, 200, 100, 50, 25, 12.5 nanomolar. Cells were transfected using RNAiMAX.

FIG. 15 is a Northern blot showing the dose response test of PMO form of PTPN1 1E2A (+5+29) (AO 33) (Diabexa-2) in IHH cells. Concentrations include 30, 15, 7.5 micromolar. Cells were transfected via nucleofection.

FIG. 16 is a Western blot showing reduction of PTP1B protein production induced by 2OMePS form of AO 33 (Diabexa-2) (400 nanomolar) and PMO form of AO 33 (Diabexa-2) (15, 7.5 micromolar) in IHH cells. Cells were harvested 72 hours after transfection by AOs.

FIG. 17 is a Northern blot showing the transfection efficiency of AO 37-41 (mouse Ptpn1 exon-2 targeting AOs) using RNAiMAX reagent in HepG2 cell line at 400 nanomolar concentration.

FIG. 18 is a Northern blot showing the transfection efficiency of AO 37-41 (mouse Ptpn1 exon-2 targeting AOs), and AO 1, 32, 33 (human PTPN1 exon-2 targeting AOs) using RNAiMAX or L3K reagent in mouse AML-12 cell line at 400 nanomolar AO concentration.

FIG. 19 is a Northern blot showing the transfection efficiency of AO 37, 38, 41 (mouse Ptpn1 exon-2 targeting AOs), and AO 1, 32, 33 (human PTPN1 exon-2 targeting AOs) using RNAiMAX in mouse AML-12 cell line. 19A: AO 37 is the mouse version of AO 1 (three mismatches), AO 38 is the mouse version of AO 32 (three mismatches), AO 41 is the mouse version of AO 33 (two mismatches); 19B: transfection efficiency of AO 37, 38, 41, 1, 32, 33 at 400 nanomolar concentration; 19C: dose dependency of AO 38; 19D: dose dependency of AO 41.

FIG. 20 is an image of the expression of PTPN1 in cancer cells. Annealing temperatures of RT-PCR reactions included 57.8° C., 60° C., and 62° C. PCR cycle was 30.

### DESCRIPTION OF INVENTION

### Detailed Description of the Invention

Antisense Oligomers

The present invention is based on the surprising discovery that altering the expression of protein tyrosine phosphatase-1B (PTP1B), coded by gene PTPN1, can mediate the effects of insulin resistance, T2DM, leptin resistance, obesity, and solid cancers. This alteration of PTP1B expression can be achieved using antisense oligomers (also known as antisense oligonucleotides, AOS, AO and AON—the terms are interchangeable).

Protein tyrosine phosphatase-1B (PTP1B) coded by gene PTPN1 is a phosphatase that negatively regulates insulin signalling and is thus responsible for insulin resistance, one of the root causes of T2DM. Apart from blocking insulin signalling, PTP1B also downregulates leptin signal transduction pathway, resulting in decreased energy expenditure and increased fat accumulation related to obesity, which contributes to insulin resistance and is one of the most important risk factors of T2DM. As PTP1B blocks both insulin and leptin signalling at the same time, the present invention has investigated the use of PTPN1 as a target gene for T2DM and obesity therapeutics development.

Without being held to any theory, the present invention is based on the understanding that:
down-regulating the expression of PTP1B leads to up-regulation of insulin signaling; and/or
down-regulating the expression of PTP1B leads to up-regulation of the leptin signal transduction pathway.

The PTP1B protein is also associated with a number of solid tumour cancers, and knockdown of PTP1B reduces cell growth, induces both cell cycle arrest and apoptosis, and reduces cancer cell migration and invasion by reversing the epithelial—mesenchymal transition (EMT) process.

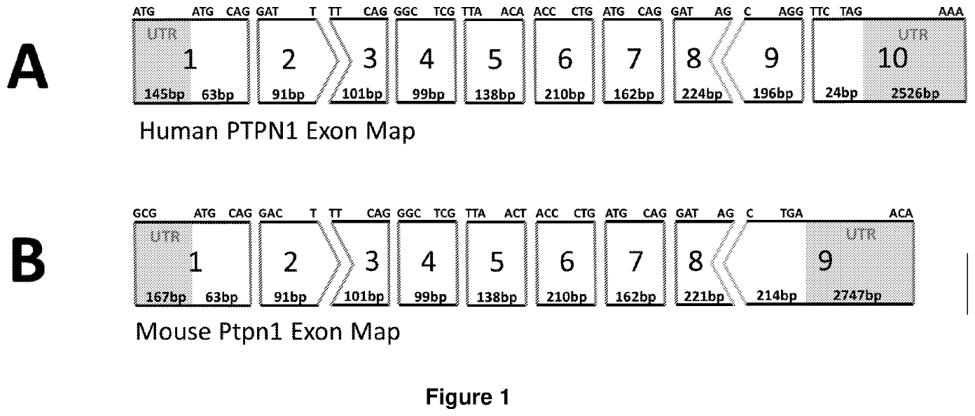

PTPN1 has ten exons, including four exons (exon 2, 3, 8, and 9) that contain residue overlap splice sites (FIG. 1). Two of those exons, exon 2 and exon 3, are near the 5′ end of the transcript. If exon 2 is skipped, pre-mature termination codons are induced in exon 3, which suggest that the variant transcript resulting from exon 2 skipping may not be translated into a functional PTP1B protein. Alternatively, exon skipping may be used to develop truncated or nonsense PTP1B proteins.

Preferably, the disease or condition treated or prevented by the antisense oligomers of the present invention is a disease that is: (i) associated with down-regulation of insulin signalling in a subject; (ii) associated with down-regulation of the leptin signal transduction pathway in a subject; and/or (iii) associated with cancer cell growth, migration and invasion. For example, the disease may be T2DM, obesity or cancer.

The present invention does not specifically seek to affect the overall expression of the PTP1B protein, for example by blocking or removing all PTPN1 transcripts. Rather, it seeks to increase the production of truncated, nonsense or prematurely terminated proteins. The overall production of PTPN1 RNA molecules may not change significantly (although some change may occur). Preferably, these truncated, nonsense or prematurely terminated proteins are lacking one or more functional domains involved in the biocatalysis process. For example, exon 1, 2, 3, 4, 5 and 6 collectively encode a tyrosine-protein phosphatase motif and translated proteins lacking this domain may not be able to catalyse the process of removing phosphate groups from phosphorylated tyrosine residues on proteins. Exon 6 and 7 encode a region containing substrate binding sites and removing these exons may generate a non-functional PTP1B protein.

The presence of internally truncated proteins (i.e. proteins lacking the amino acids encoded by one or more exons) is preferable. If the PTP1B protein is knocked out, there may be problems with elevation of PTPN1 transcription as the body tries to compensate for the reduction in the total amount of PTP1B protein. In contrast, the presence of an internally truncated protein (preferably lacking one or more of the features of the complete PTP1B protein), should be sufficient to prevent elevated transcription, but still provide a therapeutic advantage due to a reduction in the total amount of functional PTP1B protein. Preferably the exon skipping leads to skipping of exon 2; skipping of exon 2 leads to the induction of pre-mature termination codons in exon 3.

The antisense oligomer induced exon skipping of the present invention need not completely or even substantially ablate the function of the PTP1B protein. Preferably, the exon skipping process results in a reduced or compromised functionality of the PTP1B protein.

In contrast to other antisense oligomer based therapies, the present invention does not induce increased degradation of RNA via recruitment of RNase H, wherein the RNase H preferentially binds and degraded RNA bound in duplex to DNA of the PTPN1 gene. Nor does it rely on hybridization of the antisense oligomer to the PTPN1 genomic DNA or the binding of antisense oligomers to mRNA to modulate the amount of PTP1B protein produced by interfering with normal functions such as replication, transcription, translocation and translation.

Rather, the antisense oligomers are used to modify the transcription process to increase the production of truncated, nonsense or prematurely terminated proteins. Preferably, the present invention leads to skipping of exon 2, to induce pre-mature stop codons in exon 3. This would lead to a variant transcript that may not be translated into a functional PTP1B protein.

Preferably the antisense oligomers target splicing sites in the PTPN1 gene. The target site may also include some flanking sequences around the splicing sites.

The antisense oligomers may also or alternatively bind to the polyadenylation site. The target site may also be near, but not overlapping the polyadenylation site, i.e. it may instead cover sequences upstream or downstream of the polyadenylation site and in these instances the antisense oligomer may not specifically cover the polyadenylation site. Localisation to near the polyadenylation site may be sufficient to disrupt the ability of cleavage factors to bind the polyadenylation site.

According to a first aspect of the invention, there is provided antisense oligomers capable of binding to a selected target on a PTPN1 gene transcript to modify pre-mRNA splicing in a PTPN1 gene transcript or part thereof.

For example, in one aspect of the invention, there is provided an antisense oligomer of 10 to 50 nucleotides comprising a targeting sequence complementary to a region near or within the splicing sites and/or the polyadenylation site of the PTPN1 pre-mRNA.

The terms “antisense oligomer” and “antisense compound” and “antisense oligonucleotide” and “ASO” are used interchangeably and refer to a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. The cyclic subunits are based on ribose or another pentose sugar or, in a preferred embodiment, a morpholino group (see description of morpholino oligomers below). The oligomer may have exact or near sequence complementarity to the target sequence; variations in sequence near the termini of an oligomer are generally preferable to variations in the interior. The terms “pre-RNA” and “pre-mRNA” are used interchangeably.

By “isolated” is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally-occurring state, e.g., a DNA fragment that is removed from the sequences that are adjacent to the fragment in the genome. The term “isolating” as it relates to cells refers to the purification of cells (e.g., fibroblasts, lymphoblasts) from a source subject (e.g., a subject with a polynucleotide repeat disease). In the context of mRNA or protein, “isolating” refers to the recovery of mRNA or protein from a source, e.g., cells.

An antisense oligomer can be said to be “directed to” or “targeted against” a target sequence with which it hybridizes. In certain embodiments, the target sequence includes a region including splicing sites and/or the polyadenylation site and surrounding regions. The target sequence is typically a region including an AUG start codon of an mRNA, a Translation Suppressing Oligomer, or splice site of a pre-processed mRNA, a Splice Suppressing Oligomer (SSO). The target sequence for a splice site may include an mRNA sequence having its 5′ end 1 to about 25 base pairs downstream of a normal splice acceptor junction in a pre-processed mRNA. A preferred target sequence is any region of a pre-processed mRNA that includes a splice site or is contained entirely within an exon coding sequence or spans a splice acceptor or donor site. An oligomer is more generally said to be “targeted against” a biologically relevant target, such as a protein, virus, or bacteria, when it is targeted against the nucleic acid of the target in the manner described above.

As used herein, “sufficient length” refers to an antisense oligonucleotide that is complementary to at least 8, more typically 8-30, contiguous nucleobases in a target PTPN1 pre-mRNA. In some embodiments, an antisense of sufficient length includes at least 8, 9, 10, 11, 12, 13, 14, or 15 contiguous nucleobases in the target PTPN1 pre-mRNA. In other embodiments an antisense of sufficient length includes at least 16, 17, 18, 19, 20, 21, 22, 23, 24, or 25 contiguous nucleobases in the target PTPN1 pre-mRNA. An antisense oligonucleotide of sufficient length has at least a minimal number of nucleotides to be capable of specifically hybridizing to exon 2. Preferably an oligonucleotide of sufficient length is from about 10 to about 50 nucleotides in length, including oligonucleotides of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40 or more nucleotides. In one embodiment, an oligonucleotide of sufficient length is from 10 to about 30 nucleotides in length. In another embodiment, an oligonucleotide of sufficient length is from 15 to about 25 nucleotides in length. In yet another embodiment, an oligonucleotide of sufficient length is from 20 to 30, or 20 to 50, nucleotides in length. In yet another embodiment, an oligonucleotide of sufficient length is from 22 to 28, 25 to 28, 24 to 29 or 25 to 30 nucleotides in length.

In certain embodiments, the antisense oligomer has sufficient sequence complementarity to a target RNA (i.e., the RNA for which splicing factor binding site selection is modulated) to block a region of a target RNA (e.g., pre-mRNA) in an effective manner. In exemplary embodiments, such blocking of PTPN1 pre-mRNA serves to modulate or modify splicing, either by masking a binding site for a native protein that would otherwise modulate splicing and/or by altering the structure of the targeted RNA. In some embodiments, the target RNA is target pre-mRNA (e.g., PTPN1 gene pre-mRNA).

An antisense oligomer having a sufficient sequence complementarity to a target RNA sequence to modulate splicing factor binding of the target RNA means that the antisense oligomer has a sequence sufficient to trigger the masking of a binding site for a native protein that would otherwise cause truncation of the PTP1B protein and/or alters the three-dimensional structure of the targeted RNA.

Selected antisense oligomers can be made shorter, e.g., about 12 bases, or longer, e.g., about 50 bases, and include a small number of mismatches, as long as the sequence is sufficiently complementary to effect splicing factor binding modulation upon hybridization to the target sequence, and optionally forms with the RNA antisense oligomer heteroduplex having a Tm of 45° C. or greater.

Preferably, the antisense oligomer is selected from the group comprising the sequences set forth in Table 1. Preferably, the antisense oligomer is selected from the list comprising: SEQ ID NO: 1-4, 10-15, 18, 19, 23-25, 27, 29, 31-41. Preferably, the antisense oligomer used in the present invention is chosen from the list comprising: SEQ ID NO: 1, or 32-36. More preferably, the antisense oligomer used in the present invention is SEQ ID NO: 33. Preferably the antisense oligomer leads to exon skipping of exon 2.

In certain embodiments, the degree of complementarity between the target sequence and antisense oligomer is sufficient to form a stable duplex. The region of complementarity of the antisense oligomers with the target RNA sequence may be as short as 8-11 bases, but can be 12-15 bases or more, e.g., 10-50 bases, 10-40 bases, 12-30 bases, 12-25 bases, 15-25 bases, 12-20 bases, or 15-20 bases, including all integers in between these ranges. An antisense oligomer of about 16-17 bases is generally long enough to have a unique complementary sequence. In certain embodiments, a minimum length of complementary bases may be required to achieve the requisite binding Tm, as discussed herein.

In certain embodiments, oligonucleotides as long as 50 bases may be suitable, where at least a minimum number of bases, e.g., 10-12 bases, are complementary to the target sequence. In general, however, facilitated or active uptake in cells is optimized at oligonucleotide lengths of less than about 30 bases. For phosphorodiamidate morpholino oligomer (PMO) antisense oligomers described further herein, an optimum balance of binding stability and uptake generally occurs at lengths of 18-25 bases. Included are antisense oligomers (e.g., PMOs, PMO-X, PNAs, LNAs, 2′-OMe) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases.

In certain embodiments, antisense oligomers may be 100% complementary to the target sequence, or may include mismatches, e.g., to accommodate variants, as long as a heteroduplex formed between the antisense oligomer and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

Mismatches, if present, are typically less destabilizing toward the end regions of the hybrid duplex than in the middle. The number of mismatches allowed will depend on the length of the antisense oligomer, the percentage of G:C base pairs in the duplex, and the position of the mismatch(es) in the duplex, according to well understood principles of duplex stability. Although such an antisense oligomer is not necessarily 100% complementary to the target sequence, it is effective to stably and specifically bind to the target sequence, such that splicing factor binding to the target pre-mRNA is modulated.

The stability of the duplex formed between an antisense oligomer and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide Hybridization Techniques, Methods Enzymol. Vol. 154 pp. 94-107. In certain embodiments, antisense oligomers may have a binding Tm, with respect to a complementary-sequence RNA, of greater than body temperature and preferably greater than about 45° C. or 50° C. Tm's in the range 60-80° C. or greater are also included.

Additional examples of variants include antisense oligomers having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOS: 1-4, 10-15, 18, 19, 23-25, 27, 29, 31-41.

More specifically, there is provided an antisense oligomer capable of binding to a selected target site to modulate or modify splicing in a PTPN1 gene transcript or part thereof.

The antisense oligomer is preferably selected from those provided in Table 1. Preferably, the antisense oligomer is selected from the list comprising: SEQ ID NO: 1-4, 10-15, 18, 19, 23-25, 27, 29, 31-41. Preferably, the antisense oligomer used in the present invention is chosen from the list comprising: SEQ ID NO: 1, or 32-36. More preferably, the antisense oligomer used in the present invention is SEQ ID NO: 33.

The antisense oligomer induced splicing factor blockage of the present invention need not completely or even substantially reduce the amount of PTP1B produced.

TABLE 1
SEQ ID listing of antisense oligomers targeting human PTPN1 or mouse Ptpn1

| SEQ ID NO | ASO NAME | SEQUENCE |
|---|---|---|
| 1 | PTPN1 1E2A (+1+25) | AGU CAC UGG CUU CAU GUC GGA UAU C |
| 2 | PTPN1 1E3A (+19+44) | UAG UCA UUA UCU UCU UGA UGU AGU U |
| 3 | PTPN1 1E3A (+65+90) | UGU AAC UCC UUU GGG CUU CUU CCA U |
| 4 | PTPN1 1E5A (+74+99) | AUG ACU UGA UAU CUU CAG AGA UCA A |
| 5 | PTPN1 1E8A (-3+22) | GGG AAA GCU CCU UCC ACU GAU CCU G |
| 6 | PTPN1 1E8A (+76+101) | CAU UGU GUG GCU CCA GGA UUC GUU U |
| 7 | PTPN1 1E8A (+134+159) | UCU CUU CCU UCA CCC ACU GGU GAU U |
| 8 | PTPN1 1E8A (+171+196) | CGA UGC CGU AGG GUG CGG CAU UUA A |
| 9 | PTPN1 1E1D (+5-20) | GCU CCG GGG CGC UCC CGC ACC UGG U |
| 10 | PTPN1 1E2A (-20+5) | AUA UCC UAC AAA AAA GAA UAA AGA C |
| 11 | PTPN1 1E2D (+5-20) | CCG GCC ACG UGG AUA CUU ACA GGG A |
| 12 | PTPN1 1E3A (-20+5) | GUC AAC UGA AAG ACA AAC CAG AAC U |
| 13 | PTPN1 1E3D (+5-20) | AAU UCA GAC AAU CUG CUU ACC UGG G |
| 14 | PTPN1 1E4A (-20+5) | GGG CCC UGC AAA GAC ACA AUA ACA C |
| 15 | PTPN1 1E4D (+5-20) | CAA AUG AAG CCG AGA CUU ACC GAA C |
| 16 | PTPN1 1E5A (-20+5) | UUU AAC UGG GGA AAC AAA UAA UAG U |
| 17 | PTPN1 1E5D (+5-20) | GAA GUG UGU GCU AUA CUC ACU GUA A |
| 18 | PTPN1 1E6A (-20+5) | UGG GUC UGA AAG AGA AAA AUA CUC A |
| 19 | PTPN1 1E6D (+5-20) | ACC CGC GAG GGC CUC CUU ACC AGC A |
| 20 | PTPN1 1E7A (-20+5) | UCC AUC UGA AAG CCA GAG AGG AGA U |
| 21 | PTPN1 1E7D (+5-20) | CAA ACA AAG GCA AUG CUG ACC UGC A |
| 22 | PTPN1 1E8A (-20+5) | UGA UCC UUG AAA GAG CAG CAA GAG G |
| 23 | PTPN1 1E8D (+5-20) | CUG GGA CCC AAU CAU AUU ACC UUU C |
| 24 | PTPN1 1E9A (-20+5) | UCA UGC UGA GGA AUC AGA GGG CAG A |
| 25 | PTPN1 1E9D (+5-20) | GUC UGU CAG UGG AAA CAU ACC CUG U |
| 26 | PTPN1 1E10A (-20+5) | AGG AAC UGG AAU GAA ACC AAA CAG U |
| 27 | PTPN1 1E9A (+6+31) | CGA CUU CUA ACU UCA GUG UCU UGA C |
| 28 | PTPN1 1E9A (+67+92) | UGA CGG CUC CCC UUU GGC UGG GGA G |
| 29 | PTPN1 1E9A (+98+123) | UCA GUG CAU GGU CCU CGU CCU UCU C |
| 30 | PTPN1 1E9A (+164+189) | AGA GGU AAG CGC CGG CCG UGA GGA C |
| 31 | PTPN1 1E2A (+1+23) | UCA CUG GCU UCA UGU CGG AUA UC |
| 32 | PTPN1 1E2A (+3+27) | GAA GUC ACU GGC UUC AUG UCG GAU A |
| 33 | PTPN1 1E2A (+5+29) | GGG AAG UCA CUG GCU UCA UGU CGG A |
| | (Diabexa-2) | |
| 34 | PTPN1 1E2A (+7+31) | AUG GGA AGU CAC UGG CUU CAU GUC G |
| 35 | PTPN1 1E2A (+9+33) | ACA UGG GAA GUC ACU GGC UUC AUG U |
| 36 | PTPN1 1E2A (+11+35) | CUA CAU GGG AAG UCA CUG GCU UCA U |
| 37 | Ptpn1 1E2A (+1+25) | AGU CGC UGG CUU CAU GUC GAA UGU C |
| 38 | Ptpn1 1E2A (+3+27) | GAA GUC GCU GGC UUC AUG UCG AAU G |
| 39 | Ptpn1 1E2A (-5+20) | CUG GCU UCA UGU CGA AUG UCC UAC A |
| 40 | Ptpn1 1E2A (-2+23) | UCG CUG GCU UCA UGU CGA AUA UCC U |
| 41 | Ptpn1 1E2A (+5+29) | GGG AAG UCG CUG GCU UCA UGU CGA A |

Reverse complement sequence shown 5-3′. The reference point (0) set at first base of polyadenylationsignal; hence “+” refers to sequences downstream of A⁰ATAAA and “-” indicates sequences upstream

Preferably, the antisense oligomer is selected from the list comprising: SEQ ID NO: 1-4, 10-15, 18, 19, 23-25, 27, 29, 31-41. More preferably, the antisense oligomer used in the present invention is chosen from the list comprising SEQ ID NO: 1 or 32-36. Most preferably, the antisense oligomer used in the present invention is SEQ ID NO: 33.

Method of Use

The invention further provides a method for manipulating splicing factor binding in a PTPN1 gene transcript, the method including the step of:

a) providing one or more of the antisense oligomers as described herein and allowing the oligomer(s) to bind to a target nucleic acid site.

According to yet another aspect of the invention, there is provided a splicing factor binding modification target nucleic acid sequence for PTPN1 comprising the DNA equivalents of the nucleic acid sequences selected from the group consisting of SEQ ID NO: 1-4, 10-15, 18, 19, 23-25, 27, 29, 31-41, and sequences complementary thereto. Preferably the antisense oligomer leads to exon skipping of exon 2.

Designing antisense oligomers to completely mask the splicing sites and/or the polyadenylation site may not be necessary to generate a change in the proportion of truncated, nonsense or prematurely terminated proteins. Furthermore, the inventors have discovered that size or length of the antisense oligomer itself is not always a primary factor when designing antisense oligomers. With some targets, antisense oligomers as short as 20 bases were able to induce cleavage modification, in certain cases more efficiently than other longer (eg 25 bases) oligomers directed to the same region.

More specifically, the antisense oligomer may be selected from those set forth in Table 1. The sequences are preferably selected from the group consisting of any one or more of any one or more of SEQ ID NOs: 1-4, 10-15, 18, 19, 23-25, 27, 29, 31-41, and combinations or cocktails thereof. This includes sequences which can hybridise to such sequences under stringent hybridisation conditions, sequences complementary thereto, sequences containing modified bases, modified backbones, and functional truncations or extensions thereof which possess or modulate RNA processing activity in a PTPN1 gene transcript. Preferably, the ASO used in the present invention is chosen from the list comprising: SEQ ID NO: 1, or 32-36. More preferably, the antisense oligomer used in the present invention is SEQ ID NO: 33. Preferably the antisense oligomer leads to exon skipping of exon 2.

The antisense oligomer and the DNA, cDNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or pairing such that stable and specific binding occurs between the oligomer and the DNA, cDNA or RNA target. It is understood in the art that the sequence of an antisense oligomer need not be 100% complementary to that of its target sequence to be specifically hybridisable. An antisense oligomer is specifically hybridisable when binding of the compound to the target DNA or RNA molecule interferes with the normal function of the target DNA or RNA product, and there is a sufficient degree of complementarity to avoid non-specific binding of the antisense oligomer to non-target sequences under conditions in which specific binding is desired, i.e., under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0). Thus, the antisense oligomers of the present invention may include oligomers that selectively hybridise to the sequences, SEQ ID NOs: 1-4, 10-15, 18, 19, 23-25, 27, 29, 31-41 provided in Table 1.

At a given ionic strength and pH, the Tm is the temperature at which 50% of a target sequence hybridizes to a complementary polynucleotide. Such hybridization may occur with “near” or “substantial” complementarity of the antisense oligomer to the target sequence, as well as with exact complementarity.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the antisense oligomer. The length of homology comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, the antisense oligomer sequences of the invention preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, homology. Generally, the shorter the length of the antisense oligomer, the greater the homology required to obtain selective hybridisation. Consequently, where an antisense oligomer of the invention consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the antisense oligomers set out in the sequence listings herein. Nucleotide homology comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The antisense oligomer of the present invention may have regions of reduced homology, and regions of exact homology with the target sequence. It is not necessary for an oligomer to have exact homology for its entire length. For example, the oligomer may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligomer may have stretches of at least 10, 11, 12, 13, 14, 15, 16, 17, 18, 19, 20, 21, 22, 23, 24, 25 or 26 bases that are identical to the target sequence. The remaining stretches of oligomer sequence may be intermittently identical with the target sequence; for example, the remaining sequence may have an identical base, followed by a non-identical base, followed by an identical base. Alternatively (or as well) the oligomer sequence may have several stretches of identical sequence (for example 3, 4, 5 or 6 bases) interspersed with stretches of less than perfect homology. Such sequence mismatches will preferably have no or very little loss of cleavage modifying activity.

The term “modulate” or “modulates” includes to “increase” or “decrease” one or more quantifiable parameters, optionally by a defined and/or statistically significant amount. The terms “increase” or “increasing,” “enhance” or “enhancing,” or “stimulate” or “stimulating” refer generally to the ability of one or antisense oligomers or compositions to produce or cause a greater physiological response (i.e., downstream effects) in a cell or a subject relative to the response caused by either no antisense oligomer or a control compound.

By “enhance” or “enhancing,” or “increase” or “increasing,” or “stimulate” or “stimulating,” refers generally to the ability of one or antisense compounds or compositions to produce or cause a greater physiological response (i.e., downstream effects) in a cell or a subject, as compared to the response caused by either no antisense compound or a control compound. A measurable physiological response may include increased expression of a functional form of a NEAT1 protein, among other responses apparent from the understanding in the art and the description herein. An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7, 1.8, etc.) the amount produced by no antisense compound (the absence of an agent) or a control compound.

The terms “decreasing” or “decrease” refer generally to the ability of one or antisense oligomers or compositions to produce or cause a reduced physiological response (i.e., downstream effects) in a cell or a subject relative to the response caused by either no antisense oligomer or a control compound. The term “reduce” or “inhibit” may relate generally to the ability of one or more antisense compounds of the invention to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a PTP1B related condition. A “decrease” in a response may be statistically significant as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

Relevant physiological or cellular responses (in vivo or in vitro) will be apparent to persons skilled in the art, and may include decreases in the amount of PTP1B protein. An “increased” or “enhanced” amount is typically a statistically significant amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7. 1.8) the amount produced by no antisense oligomer (the absence of an agent) or a control compound. The term “reduce” or “inhibit” may relate generally to the ability of one or more antisense oligomers or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a disease associated with T2DM such as insulin resistance and leptin resistance, or a disease such as cancer. A “decrease” in a response may be statistically significant as compared to the response produced by no antisense oligomer or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The length of an antisense oligomer may vary, as long as it is capable of binding selectively to the intended location within the pre-mRNA molecule. The length of such sequences can be determined in accordance with selection procedures described herein. Generally, the antisense oligomer will be from about 10 nucleotides in length, up to about 50 nucleotides in length. It will be appreciated, however, that any length of nucleotides within this range may be used in the method. Preferably, the length of the antisense oligomer is between 10 and 40, 10 and 35, 15 to 30 nucleotides in length or 20 to 30 nucleotides in length, most preferably about 25 to 30 nucleotides in length. For example, the oligomer may be 20, 21, 22, 23, 24, 25, 26, 27, 28, 29 or 30 nucleotides in length.

As used herein, an “antisense oligomer” or “ASO” refers to a linear sequence of nucleotides, or nucleotide analogues, that allows the nucleobase to hybridize to a target sequence in an RNA by Watson-Crick base pairing, to form an oligonucleotide:RNA heteroduplex within the target sequence. The terms “antisense oligomer”, “antisense oligonucleotide”, “oligomer” and “antisense compound” may be used interchangeably to refer to an oligonucleotide. The cyclic subunits may be based on ribose or another pentose sugar or, in certain embodiments, a morpholino group (see description of morpholino oligonucleotides below). Also contemplated are peptide nucleic acids (PNAs), locked nucleic acids (LNAs), and 2′-O-Methyl (2′-OMe) oligonucleotides, among other antisense agents known in the art.

In some embodiments, the antisense oligonucleotides have the chemical composition of a naturally occurring nucleic acid molecule, i.e., the antisense oligonucleotides do not include a modified or substituted base, sugar, or inter-subunit linkage.

In a preferred embodiment, the antisense oligonucleotides of the present invention are non-naturally occurring nucleic acid molecules, or “oligonucleotide analogues”. For example, non-naturally occurring nucleic acids can include one or more non-natural base, sugar, and/or inter-subunit linkage, e.g., a base, sugar, and/or linkage that has been modified or substituted with respect to that found in a naturally occurring nucleic acid molecule. Exemplary modifications are described below. In some embodiments, non-naturally occurring nucleic acids include more than one type of modification, e.g. sugar and base modifications, sugar and linkage modifications, base and linkage modifications, or base, sugar, and linkage modifications. For example, in some embodiments, the antisense oligonucleotides contain a non-natural (e.g.

modified or substituted) base. In some embodiments, the antisense oligonucleotides contain a non-natural (e.g. modified or substituted) sugar. In some embodiments, the antisense oligonucleotides contain a non-natural (e.g. modified or substituted) inter-subunit linkage. In some embodiments, the antisense oligonucleotides contain more than one type of modification or substitution, e.g. a non-natural base and/or a non-natural sugar, and/or a non-natural inter-subunit linkage.

Thus included are non-naturally-occurring antisense oligomers having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligonucleotide analogues support bases capable of hydrogen bonding by Watson-Crick base pairing to standard polynucleotide bases, where the analogue backbone presents the bases in a manner to permit such hydrogen bonding in a sequence-specific fashion between the oligonucleotide analogue molecule and bases in a standard polynucleotide (e.g., single-stranded RNA or single-stranded DNA). Preferred analogues are those having a substantially uncharged, phosphorus containing backbone.

One method for producing antisense oligomers is the methylation of the 2′ hydroxyribose position and the incorporation of a phosphorothioate backbone produces molecules that superficially resemble RNA but that are much more resistant to nuclease degradation, although persons skilled in the art of the invention will be aware of other forms of suitable backbones that may be useable in the objectives of the invention.

To avoid degradation of pre-RNA during duplex formation with the antisense oligomers, the antisense oligomers used in the method may be adapted to minimise or prevent cleavage by endogenous RNase H. Antisense molecules that do not activate RNase H can be made in accordance with known techniques (see, e.g., U.S. Pat. No. 5,149,797). Such antisense molecules, which may be deoxyribonucleotide or ribonucleotide sequences, simply contain any structural modification which sterically hinders or prevents binding of RNase H to a duplex molecule containing the oligonucleotide as one member thereof, which structural modification does not substantially hinder or disrupt duplex formation. Because the portions of the oligonucleotide involved in duplex formation are substantially different from those portions involved in RNase H binding thereto, numerous antisense molecules that do not activate RNase H are available. This property is highly preferred, as the treatment of the RNA with the unmethylated oligomers, either intracellular or in crude extracts that contain RNase H, leads to degradation of the pre-mRNA:antisense oligomer duplexes. Any form of modified antisense oligomers that is capable of by-passing or not inducing such degradation may be used in the present method. The nuclease resistance may be achieved by modifying the antisense oligomers of the invention so that it comprises partially unsaturated aliphatic hydrocarbon chain and one or more polar or charged groups including carboxylic acid groups, ester groups, and alcohol groups.

An example of antisense oligomers which when duplexed with RNA are not cleaved by cellular RNase H is 2′-O-methyl derivatives. Such 2′-O-methyl-oligoribonucleotides are stable in a cellular environment and in animal tissues, and their duplexes with RNA have higher Tm values than their ribo- or deoxyribo-counterparts. Alternatively, the nuclease resistant antisense oligomers of the invention may have at least one of the last 3′-terminus nucleotides fluoridated. Still alternatively, the nuclease resistant antisense oligomers of the invention have phosphorothioate bonds linking between at least two of the last 3-terminus nucleotide bases, preferably having phosphorothioate bonds linking between the last four 3′-terminal nucleotide bases.

Modified or modulated RNA splicing may also be achieved with alternative oligonucleotide chemistry (see, e.g., U.S. Pat. No. 5,149,797). For example, the antisense oligomer may be chosen from the list comprising: phosphoramidate or phosphorodiamidate morpholino oligomer (PMO); PMO-X; PPMO; peptide nucleic acid (PNA); a locked nucleic acid (LNA) and derivatives including alpha-L-LNA, 2′-amino LNA, 4′-methyl LNA and 4′-O-methyl LNA; ethylene bridged nucleic acids (ENA) and their derivatives; phosphorothioate oligomer; tricyclo-DNA oligomer (tcDNA); tricyclophosphorothioate oligomer; 2′O-Methyl-modified oligomer (2′-OMe); 2′-O-methoxy ethyl (2′-MOE); 2′-fluoro, 2′-fluroarabino (FANA); unlocked nucleic acid (UNA); hexitol nucleic acid (HNA); cyclohexenyl nucleic acid (CeNA); 2′-amino (2′-NH₂); 2′-O-ethyleneamine or any combination of the foregoing as mixmers or as gapmers.

To further improve the delivery efficacy, the abovementioned modified nucleotides are often conjugated with fatty acids/lipid/cholesterol/amino acids/carbohydrates/polysaccharides/nanoparticles etc. to the sugar or nucleobase moieties. These conjugated nucleotide derivatives can also be used to construct antisense oligomers to modify cleavage factor binding. Antisense oligomer-induced splicing factor binding modification of the PTPN1 gene transcripts have generally used either oligoribonucleotides, PNAs, 2′OMe or MOE modified bases on a phosphorothioate backbone. Although 2′OMe ASOs are used for oligo design, due to their efficient uptake in vitro when delivered as cationic lipoplexes, these compounds are susceptible to nuclease degradation and are not considered ideal for in vivo or clinical applications. When alternative chemistries are used to generate the antisense oligomers of the present invention, the uracil (U) of the sequences provided herein may be replaced by a thymine (T).

For example, such antisense molecules may be oligonucleotides wherein at least one, or all, of the inter-nucleotide bridging phosphate residues are modified phosphates, such as methyl phosphonates, methyl phosphorothioates, phosphoromorpholidates, phosphoropiperazidates and phosphor amidates. For example, every other one of the internucleotide bridging phosphate residues may be modified as described. In another non-limiting example, such antisense molecules are molecules wherein at least one, or all, of the nucleotides contain a 2′ lower alkyl moiety (e.g., Ci-C4, linear or branched, saturated or unsaturated alkyl, such as methyl, ethyl, ethenyl, propyl, 1-propenyl, 2-propenyl, and isopropyl). For example, every other one of the nucleotides may be modified as described.

Specific examples of antisense oligonucleotides useful in this invention include oligonucleotides containing modified backbones or non-natural inter-subunit linkages.

Oligonucleotides having modified backbones include those that retain a phosphorus atom in the backbone and those that do not have a phosphorus atom in the backbone. Modified oligonucleotides that do not have a phosphorus atom in their inter-nucleoside backbone can also be considered to be oligonucleosides.

In other antisense molecules, both the sugar and the inter-nucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleo-bases are retained and are bound directly or indirectly to aza nitrogen atoms of the amide portion of the backbone.

Modified oligonucleotides may also contain one or more substituted sugar moieties. Oligonucleotides may also include nucleobase (often referred to in the art simply as “base”) modifications or substitutions. Oligonucleotides containing a modified or substituted base include oligonucleotides in which one or more purine or pyrimidine bases most commonly found in nucleic acids are replaced with less common or non-natural bases.

Purine bases comprise a pyrimidine ring fused to an imidazole ring; adenine and guanine are the two purine nucleobases most commonly found in nucleic acids. These may be substituted with other naturally-occurring purines, including but not limited to N₆-methyladenine, N₂-methylguanine, hypoxanthine, and 7-methylguanine.

Pyrimidine bases comprise a six-membered pyrimidine ring; cytosine, uracil, and thymine are the pyrimidine bases most commonly found in nucleic acids. These may be substituted with other naturally-occurring pyrimidines, including but not limited to 5-methylcytosine, 5-hydroxymethylcytosine, pseudouracil, and 4-thiouracil. In one embodiment, the oligonucleotides described herein contain thymine bases in place of uracil.

Other modified or substituted bases include, but are not limited to, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N₂-cyclopentylguanine (cPent-G), N₂-cyclopentyl-2-aminopurine (cPent-AP), and N₂-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). Examples of derivatives of Super A, Super G and Super T can be found in U.S. Pat. No. 6,683,173 (Epoch Biosciences). cPent-G, cPent-AP and Pr-AP were shown to reduce immunostimulatory effects when incorporated in siRNA (Peacock H. et al. J. Am. Chem. Soc. 2011, 133, 9200). Pseudouracil is a naturally occurring isomerized version of uracil, with a C-glycoside rather than the regular N-glycoside as in uridine. Pseudouridine-containing synthetic mRNA may have an improved safety profile compared to uridine-containing mPvNA (see WO 2009127230).

Certain modified or substituted nucleo-bases are particularly useful for increasing the binding affinity of the antisense oligonucleotides of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

In some embodiments, modified or substituted nucleo-bases are useful for facilitating purification of antisense oligonucleotides. For example, in certain embodiments, antisense oligonucleotides may contain three or more (e.g., 3, 4, 5, 6 or more) consecutive guanine bases. In certain antisense oligonucleotides, a string of three or more consecutive guanine bases can result in aggregation of the oligonucleotides, complicating purification. In such antisense oligonucleotides, one or more of the consecutive guanines can be substituted with inosine. The substitution of inosine for one or more guanines in a string of three or more consecutive guanine bases can reduce aggregation of the antisense oligonucleotide, thereby facilitating purification.

In one embodiment, another modification of the antisense oligonucleotides involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

It is not necessary for all positions in a given compound to be uniformly modified, and in fact more than one of the aforementioned modifications may be incorporated in a single compound or even at a single nucleoside within an oligonucleotide. The present invention also includes antisense oligonucleotides that are chimeric compounds. “Chimeric” antisense compounds or “chimeras,” in the context of this invention, are antisense molecules, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the increased resistance to nuclease degradation, increased cellular uptake, and an additional region for increased binding affinity for the target nucleic acid.

The antisense molecules used in accordance with this invention may be conveniently and routinely made through the well-known technique of solid phase synthesis. Equipment for such synthesis is sold by several vendors including, for example, Applied Biosystems (Foster City, Calif.). One method for synthesising oligonucleotides on a modified solid support is described in U.S. Pat. No. 4,458,066.

In another non-limiting example, such antisense oligomers are molecules wherein at least one, or all, of the nucleotides contain a 2′ lower alkyl moiety (such as, for example, C1-C4, linear or branched, saturated or unsaturated alkyl, such as methyl, ethyl, ethenyl, propyl, 1-propenyl, 2-propenyl, and isopropyl). For example, every other one of the nucleotides may be modified as described.

While the antisense oligomers described above are a preferred form of the antisense oligomers of the present invention, the present invention includes other oligomeric antisense molecules, including but not limited to oligomer mimetics such as are described below.

Another preferred chemistry is the phosphorodiamidate morpholino oligomer (PMO) oligomeric compounds, which are not degraded by any known nuclease or protease. These compounds are uncharged, do not activate RNase H activity when bound to a RNA strand and have been shown to exert sustained cleavage factor binding modulation after in vivo administration (Summerton and Weller, Antisense Nucleic Acid Drug Development, 7, 187-197).

Modified oligomers may also contain one or more substituted sugar moieties. Oligomers may also include nucleobase (often referred to in the art simply as “base”) modifications or substitutions. Certain nucleobases are particularly useful for increasing the binding affinity of the oligomeric compounds of the invention. These include 5-substituted pyrimidines, 6-azapyrimidines, and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C., even more particularly when combined with 2′-O-methoxyethyl sugar modifications. In one embodiment, at least one pyrimidine base of the oligonucleotide comprises a 5-substituted pyrimidine base, wherein the pyrimidine base is selected from the group consisting of cytosine, thymine and uracil. In one embodiment, the 5-substituted pyrimidine base is 5-methylcytosine. In another embodiment, at least one purine base of the oligonucleotide comprises an N-2, N-6 substituted purine base. In one embodiment, the N-2, N-6 substituted purine base is 2, 6-diaminopurine.

In one embodiment, the antisense oligonucleotide includes one or more 5-methylcytosine substitutions alone or in combination with another modification, such as 2′-O-methoxyethyl sugar modifications. In yet another embodiment, the antisense oligonucleotide includes one or more 2, 6-diaminopurine substitutions alone or in combination with another modification.

In some embodiments, the antisense oligonucleotide is chemically linked to one or more moieties, such as a polyethylene glycol moiety, or conjugates, such as an arginine-rich cell penetrating peptide that enhance the activity, cellular distribution, or cellular uptake of the antisense oligonucleotide. In one exemplary embodiment, the arginine-rich polypeptide is covalently coupled at its N-terminal or C-terminal residue to the 3′ or 5′ end of the antisense compound. Also in an exemplary embodiment, the antisense compound is composed of morpholino subunits and phosphorus-containing inter-subunit linkages joining a morpholino nitrogen of one subunit to a 5′ exocyclic carbon of an adjacent subunit.

In another aspect, the invention provides expression vectors that incorporate the antisense oligonucleotides described above, e.g., the antisense oligonucleotides of SEQ ID NOs: 1-41. In some embodiments, the expression vector is a modified retrovirus or non-retroviral vector, such as an adeno-associated viral vector.

Another modification of the oligomers of the invention involves chemically linking to the oligomer one or more moieties or conjugates that enhance the activity, cellular distribution or cellular uptake of the oligomer. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, myristyl, or an octadecylamine or hexylamino-carbonyl-oxycholesterol moiety.

Cell penetrating peptides have been added to phosphorodiamidate morpholino oligomers to enhance cellular uptake and nuclear localization. Different peptide tags have been shown to influence efficiency of uptake and target tissue specificity, as shown in Jearawiriyapaisarn et al. (2008), Mol. Ther. 16 9, 1624-1629. The terms “cell penetrating peptide” and “CPP” are used interchangeably and refer to cationic cell penetrating peptides, also called transport peptides, carrier peptides, or peptide transduction domains. The peptides, as shown herein, have the capability of inducing cell penetration within 100% of cells of a given cell culture population and allow macromolecular translocation within multiple tissues in vivo upon systemic administration.

It is not necessary for all positions in a given compound to be uniformly modified, and in fact more than one of the aforementioned modifications may be incorporated in a single compound or even at a single nucleoside within an oligomer. The present invention also includes antisense oligomers that are chimeric compounds. “Chimeric” antisense oligomers or “chimeras,” in the context of this invention, are antisense oligomers, particularly oligomers, which contain two or more chemically distinct regions, each made up of at least one monomer unit, i.e., a nucleotide in the case of an oligomer compound. These oligomers typically contain at least one region wherein the oligomer is modified so as to confer upon the oligomer or antisense oligomer increased resistance to nuclease degradation, increased cellular uptake, and an additional region for increased binding affinity for the target nucleic acid.

The activity of antisense oligomers and variants thereof can be assayed according to routine techniques in the art. For example, isoform forms and expression levels of surveyed RNAs and proteins may be assessed by any of a wide variety of well-known methods for detecting isoforms and/or expression of a transcribed nucleic acid or protein. Non-limiting examples of such methods include RT-PCR of isoforms of RNA followed by size separation of PCR products, nucleic acid hybridization methods e.g., Northern blots and/or use of nucleic acid arrays; fluorescent in situ hybridization to detect RNA transcripts inside cells; nucleic acid amplification methods; immunological methods for detection of proteins; protein purification methods; and protein function or activity assays.

RNA expression levels can be assessed by preparing RNA/cDNA (i.e., a transcribed polynucleotide) from a cell, tissue or organism, and by hybridizing the RNA/cDNA with a reference polynucleotide, which is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcripT1(s).

The present invention provides antisense oligomer modified splicing factor binding of the PTPN1 gene transcript, clinically relevant oligomer chemistries and delivery systems to direct reduction of full length PTPN1 transcript to therapeutic levels. Substantial changes in the amount of PTPN1 RNA are achieved by:
1) oligomer refinement in vitro using cell lines, through experimental assessment of (i) modification of splicing factor binding target motifs, (ii) antisense oligomer length and development of oligomer cocktails, (iii) choice of chemistry, and (iv) the addition of cell-penetrating peptides (CPP) to enhance oligomer delivery; and
2) detailed evaluation of a novel approach to decrease PTPN1 transcripts.

As such, it is demonstrated herein that processing of PTPN1 RNA can be manipulated with specific antisense oligomers. In this way functionally significant decreases in the amount of the PTP1B protein can be obtained, thereby reducing the pathology of a disease associated with PTP1B.

Preferably, the disease associated with PTP1B is a disease that is: (i) associated with down-regulation of insulin signalling in a subject; (ii) associated with down-regulation of the leptin signal transduction pathway in a subject; and/or (iii) associated with cancer cell growth, migration and invasion. For example, the disease may be T2DM and/or obesity. In addition, the disease may be solid cancers.

The antisense oligomers used in accordance with this invention may be conveniently made through the well-known technique of solid phase synthesis. Equipment for such synthesis is sold by several vendors including, for example, Applied Biosystems (Foster City, Calif.). One method for synthesising oligomers on a modified solid support is described in U.S. Pat. No. 4,458,066.

Any other means for such synthesis known in the art may additionally or alternatively be employed. It is well known to use similar techniques to prepare oligomers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The antisense oligomers of the invention are synthesised in vitro and do not include antisense compositions of biological origin, or genetic vector constructs designed to direct the in vivo synthesis of antisense oligomers. The molecules of the invention may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

Also included are vector delivery systems that are capable of expressing the oligomeric, NEAT1-targeting sequences of the present invention, such as vectors that express a polynucleotide sequence comprising any one or more of SEQ ID NOs: 1-4, 10-15, 18, 19, 23-25, 27, 29, 31-41, as described herein. By “vector” or “nucleic acid construct” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated.

Method of Treatment

The antisense oligomers of the present invention also can be used as a prophylactic or therapeutic, which may be utilised for the purpose of treatment of a disease. Accordingly, in one embodiment the present invention provides antisense oligomers that bind to a selected target in the PTPN1 RNA to modify splicing of the RNA as described herein, in a therapeutically effective amount, admixed with a pharmaceutically acceptable carrier, diluent, or excipient.

An “effective amount” or “therapeutically effective amount” refers to an amount of therapeutic compound, such as an antisense oligomer, administered to a mammalian subject, either as a single dose or as part of a series of doses, which is effective to produce a desired therapeutic effect.

The invention therefore provides a pharmaceutical, prophylactic, or therapeutic composition to treat, prevent or ameliorate the effects of a disease associated with PTP1B in a subject, the composition comprising:
a) one or more antisense oligomers as described herein, and
b) one or more pharmaceutically acceptable carriers and/or diluents.

Preferably, the disease associated with PTP1B is a disease that is: (i) associated with down-regulation of insulin signalling in a subject; (ii) associated with down-regulation of the leptin signal transduction pathway in a subject; and/or (iii) associated with cancer cell growth, migration and invasion. For example, the disease may be T2DM, obesity or solid cancers.

Preferably, the antisense oligomer used in the present invention is chosen from the list comprising:
SEQ ID NO: 1-4, 10-15, 18, 19, 23-25, 27, 29, 31-41;
SEQ ID NO: 1 or 32-36; or
SEQ ID NO: 33.

Preferably the antisense oligomer leads to exon skipping of exon 2.

The composition may comprise about 1 nM to 1000 nM of each of the desired antisense oligomer(s) of the invention. Preferably, the composition may comprise about 1 nM to 500 nM, 10 nM to 500 nM, 50 nM to 750 nM, 10 nM to 500 nM, 1 nM to 100 nM, 1 nM to 50 nM, 1 nM to 40 nM, 1 nM to 30 nM, 1 nM to 20 nM, most preferably between 1 nM and 10 nM of each of the antisense oligomer(s) of the invention.

The composition may comprise about 1 nm, 2 nm, 3 nm, 4 nm, 5 nm, 6 nm, 7 nm, 8 nm, 9 nm, 10 nm, 20 nm, 50 nm, 75 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm or 1000 nm of each of the desired antisense oligomer(s) of the invention.

The present invention further provides one or more antisense oligomers adapted to aid in the prophylactic or therapeutic treatment, prevention or amelioration of symptoms of a disease or pathology associated with PTP1B in a form suitable for delivery to a subject.

The phrase “pharmaceutically acceptable” refers to molecular entities and compositions that are physiologically tolerable and do not typically produce an allergic or similarly untoward reaction, such as gastric upset and the like, when administered to a subject. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Martin, Remington's Pharmaceutical Sciences, 18th Ed., Mack Publishing Co., Easton, Pa., (1990).

Pharmaceutical Compositions

In a form of the invention there are provided pharmaceutical compositions comprising therapeutically effective amounts of one or more antisense oligomers of the invention together with pharmaceutically acceptable diluents, preservatives, solubilizers, emulsifiers, adjuvants, and/or carriers. Such compositions include diluents of various buffer content (e.g. Tris-HCl, acetate, phosphate), pH and ionic strength and additives such as detergents and solubilizing agents (e.g. Tween 80, Polysorbate 80), anti-oxidants (e.g., ascorbic acid, sodium metabisulfite), preservatives (e.g. Thimersol, benzyl alcohol) and bulking substances (e.g., lactose, mannitol). The material may be incorporated into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, for example, Martin, Remington's Pharmaceutical Sciences, 18th Ed. (1990, Mack Publishing Co., Easton, Pa. 18042) pages 1435-1712 that are herein incorporated by reference. The compositions may be prepared in liquid form, or may be in dried powder, such as a lyophilised form.

It will be appreciated that pharmaceutical compositions provided according to the present invention may be administered by any means known in the art. Preferably, the pharmaceutical compositions for administration are administered by injection, orally, topically or by the pulmonary or nasal route. The antisense oligomers are more preferably delivered by intravenous, intra-arterial, intraperitoneal, intramuscular or subcutaneous routes of administration. The appropriate route may be determined by one of skill in the art, as appropriate to the condition of the subject under treatment. Vascular or extravascular circulation, the blood or lymph system, and the cerebrospinal fluid are some non-limiting sites where the antisense oligomer may be introduced. Direct CNS delivery may be employed, for instance, intracerebral ventribular or intrathecal administration may be used as routes of administration.

Formulations for topical administration include those in which the oligomers of the disclosure are in admixture with a topical delivery agent such as lipids, liposomes, fatty acids, fatty acid esters, steroids, chelating agents and surfactants. Lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). For topical or other administration, oligomers of the disclosure may be encapsulated within liposomes or may form complexes thereto, in particular to cationic liposomes. Alternatively, oligomers may be complexed to lipids, in particular to cationic lipids. Fatty acids and esters, pharmaceutically acceptable salts thereof, and their uses are further described in U.S. Pat. No. 6,287,860 and/or U.S. patent application Ser. No. 09/315,298 filed on May 20, 1999.

In certain embodiments, the antisense oligomers of the disclosure can be delivered by transdermal methods (e.g., via incorporation of the antisense oligomers into, e.g., emulsions, with such antisense oligomers optionally packaged into liposomes). Such transdermal and emulsion/liposome-mediated methods of delivery are described for delivery of antisense oligomers in the art, e.g., in U.S. Pat. No. 6,965,025.

The antisense oligomers described herein may also be delivered via an implantable device. Design of such a device is an art-recognized process, with, e.g., synthetic implant design described in, e.g., U.S. Pat. No. 6,969,400.

Compositions and formulations for oral administration include powders or granules, microparticulates, nanoparticulates, suspensions or solutions in water or non-aqueous media, capsules, gel capsules, sachets, tablets or minitablets. Thickeners, flavouring agents, diluents, emulsifiers, dispersing aids or binders may be desirable. Oral formulations are those in which oligomers of the disclosure are administered in conjunction with one or more penetration enhancers surfactants and chelators. Surfactants include fatty acids and/or esters or salts thereof, bile acids and/or salts thereof. Bile acids/salts and fatty acids and their uses are further described in U.S. Pat. No. 6,287,860. In some embodiments, the present disclosure provides combinations of penetration enhancers, for example, fatty acids/salts in combination with bile acids/salts. An exemplary combination is the sodium salt of lauric acid, capric acid and UDCA. Further penetration enhancers include polyoxyethylene-9-lauryl ether, polyoxyethylene-20-cetyl ether. Oligomers of the disclosure may be delivered orally, in granular form including sprayed dried particles, or complexed to form micro or nanoparticles. Oligomer complexing agents and their uses are further described in U.S. Pat. No. 6,287,860. Oral formulations for oligomers and their preparation are described in detail in U.S. Pat. No. 6,887,906, U.S. Ser. No. 09/315,298 filed May 20, 1999 and/or US20030027780.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

The delivery of a therapeutically useful amount of antisense oligomers may be achieved by methods previously published. For example, intracellular delivery of the antisense oligomer may be via a composition comprising an admixture of the antisense oligomer and an effective amount of a block copolymer. An example of this method is described in US patent application US20040248833. Other methods of delivery of antisense oligomers to the nucleus are described in Mann C J et al. (2001) Proc. Natl. Acad. Science, 98(1) 42-47, and in Gebski et al. (2003) Human Molecular Genetics, 12(15): 1801-1811. A method for introducing a nucleic acid molecule into a cell by way of an expression vector either as naked DNA or complexed to lipid carriers, is described in U.S. Pat. No. 6,806,084.

In certain embodiments, the antisense oligomers of the invention and therapeutic compositions comprising the same can be delivered by transdermal methods (e.g., via incorporation of the antisense oligomers into, e.g., emulsions, with such antisense oligomers optionally packaged into liposomes). Such transdermal and emulsion/liposome-mediated methods of delivery are described for delivery of antisense oligomers in the art, e.g., in U.S. Pat. No. 6,965,025.

It may be desirable to deliver the antisense oligomer in a colloidal dispersion system. Colloidal dispersion systems include macromolecule complexes, nanocapsules, microspheres, beads, and lipid-based systems including oil-in-water emulsions, micelles, mixed micelles, and liposomes or liposome formulations. These colloidal dispersion systems can be used in the manufacture of therapeutic pharmaceutical compositions.

Liposomes are artificial membrane vesicles, which are useful as delivery vehicles in vitro and in vivo. These formulations may have net cationic, anionic, or neutral charge characteristics and have useful characteristics for in vitro, in vivo and ex vivo delivery methods. It has been shown that large unilamellar vesicles can encapsulate a substantial percentage of an aqueous buffer containing large macromolecules. RNA and DNA can be encapsulated within the aqueous interior and be delivered to cells in a biologically active form (Fraley, et al., Trends Biochem. Sci. 6:77, 1981).

In order for a liposome to be an efficient gene transfer vehicle, the following characteristics should be present: (1) encapsulation of the antisense oligomer of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988). The composition of the liposome is usually a combination of phospholipids, particularly high phase-transition-temperature phospholipids, usually in combination with steroids, especially cholesterol. Other phospholipids or other lipids may also be used. The physical characteristics of liposomes depend on pH, ionic strength, and the presence of divalent cations. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The antisense oligomers described herein may also be delivered via an implantable device. Design of such a device is an art-recognized process, with, e.g., synthetic implant design described in, e.g., U.S. Pat. No. 6,969,400, the contents of which are incorporated in their entirety by reference herein.

Antisense oligomers can be introduced into cells using art-recognized techniques (e.g., transfection, electroporation, fusion, liposomes, colloidal polymeric particles and viral and non-viral vectors as well as other means known in the art). The method of delivery selected will depend at least on the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

As known in the art, antisense oligomers may be delivered using, for example, methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (refer to Dokka and Rojanasakul, Advanced Drug Delivery Reviews 44, 35-49, incorporated by reference in its entirety).

The antisense oligomer may also be combined with other pharmaceutically acceptable carriers or diluents to produce a pharmaceutical composition. Suitable carriers and diluents include isotonic saline solutions, for example phosphate-buffered saline. The composition may be formulated for parenteral, intramuscular, intravenous, subcutaneous, intraocular, oral, or transdermal administration.

The routes of administration described are intended only as a guide since a skilled practitioner will be able to readily determine the optimum route of administration and any dosage for any particular animal and condition.

Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992) 256:808-813; Barteau et al. (2008), Curr Gene Ther; 8(5):313-23; Mueller et al. (2008). Clin Rev Allergy Immunol; 35(3):164-78; Li et al. (2006) Gene Ther., 13(18):1313-9; Simoes et al. (2005) Expert Opin Drug Deliv; 2(2):237-54.

The antisense oligomers of the invention encompass any pharmaceutically acceptable salts, esters, or salts of such esters, or any other compound which, upon administration to an animal including a human, is capable of providing (directly or indirectly) the biologically active metabolite or residue thereof. Accordingly, as an example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention: i.e. salts that retain the desired biological activity of the parent compound and do not impart undesired toxicological effects thereto. For oligomers, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The pharmaceutical compositions of the present invention may be administered in a number of ways depending upon whether local or systemic treatment is desired and upon the area to be treated. Administration may be topical (including ophthalmic and mucous membranes, as well as rectal delivery), pulmonary, e.g., by inhalation or insufflation of powders or aerosols (including by nebulizer, intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration. Oligomers with at least one 2′-O-methoxyethyl modification are believed to be particularly useful for oral administration. Preferably, the antisense oligomer is delivered via the subcutaneous or intravenous route.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipienT1(s). In general, the formulations are prepared by uniformly and intimately bringing into association the active ingredients with liquid carriers or finely divided solid carriers or both, and then, if necessary, shaping the product.

Administration

In one embodiment, the antisense oligomer is administered in an amount and manner effective to result in a peak blood concentration of at least 200-400 nM antisense oligomer. Typically, one or more doses of antisense oligomer are administered, generally at regular intervals, for a period of about one to two weeks. Preferred doses for oral administration are from about 1 mg to 1000 mg oligomer per 70 kg. In some cases, doses of greater than 1000 mg oligomer/subject may be necessary. For intra venous administration, preferred doses are from about 0.5 mg to 1000 mg oligomer per 70 kg. For intra venous or sub cutaneous administration, the antisense oligomer may be administered at a dosage of about 120 mg/kg daily or weekly.

The antisense oligomer may be administered at regular intervals for a short time period, e.g., daily for two weeks or less. However, in some cases the oligomer is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests and physiological examination of the subject under treatment.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the subject. Persons of ordinary skill can easily determine optimum dosages, dosing methodologies and repetition rates. Optimum dosages may vary depending on the relative potency of individual oligomers, and can generally be estimated based on EC50s found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligomer is administered in maintenance doses, ranging from 0.01 μg to 100 g per kg of body weight, once or more daily, to once every 20 years.

An effective in vivo treatment regimen using the antisense oligomers of the invention may vary according to the duration, dose, frequency and route of administration, as well as the condition of the subject under treatment (i.e., prophylactic administration versus administration in response to localized or systemic infection). Accordingly, such in vivo therapy will often require monitoring by tests appropriate to the particular type of disorder under treatment, and corresponding adjustments in the dose or treatment regimen, in order to achieve an optimal therapeutic outcome.

Treatment may be monitored, e.g., by general indicators of disease known in the art. As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or condition associated with PTP1B, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

A “subject,” as used herein, includes any animal that exhibits a symptom, or is at risk for exhibiting a symptom, which can be treated with an antisense compound of the invention, or any of the symptoms associated with these conditions (e.g. down-regulation of insulin signalling, down-regulation of the leptin signal transduction pathway, reduction in cancer cell growth, migration and invasion). Suitable subjects include laboratory animals (such as mouse, rat, rabbit, or guinea pig), farm animals, and domestic animals or pets (such as a cat or dog). Non-human primates and, preferably, human subjects, are included.

The efficacy of an in vivo administered antisense oligomers of the invention may be determined from biological samples (tissue, blood, urine etc.) taken from a subject prior to, during and subsequent to administration of the antisense oligomer. Assays of such samples include (1) monitoring the presence or absence of heteroduplex formation with target and non-target sequences, using procedures known to those skilled in the art, e.g., an electrophoretic gel mobility assay; (2) monitoring the amount of a mutant RNA in relation to a reference normal RNA or protein as determined by standard techniques such as RT-PCR, Northern blotting, ELISA or Western blotting.

Intranuclear oligomer delivery is a major challenge for antisense oligomers. Different cell-penetrating peptides (CPP) localize PMOs to varying degrees in different conditions and cell lines, and novel CPPs have been evaluated by the inventors for their ability to deliver PMOs to the target cells. The terms CPP or “a peptide moiety which enhances cellular uptake” are used interchangeably and refer to cationic cell penetrating peptides, also called “transport peptides”, “carrier peptides”, or “peptide transduction domains”. The peptides, as shown herein, have the capability of inducing cell penetration within about or at least about 30%, 40%, 50%, 60%, 70%, 80%, 90%, or 100% of cells of a given cell culture population and allow macromolecular translocation within multiple tissues in vivo upon systemic administration. CPPs are well-known in the art and are disclosed, for example in U.S. Application No. 2010/0016215, which is incorporated by reference in its entirety.

The present invention therefore provides antisense oligomers of the present invention in combination with cell-penetrating peptides for manufacturing therapeutic pharmaceutical compositions.

According to a still further aspect of the invention, there is provided one or more antisense oligomers as described herein for use in an antisense oligomer-based therapy. Preferably, the therapy is for a disease associated with PTP1B.

Preferably, the disease associated with PTP1B is a disease that is: (i) associated with down-regulation of insulin signaling in a subject; (ii) associated with down-regulation of the leptin signal transduction pathway in a subject; and/or (iii) associated with cancer cell growth, migration and invasion. For example, the disease may be T2DM and/or obesity. Alternatively, the disease may be solid cancers.

More specifically, the antisense oligomer may be selected from the group consisting of any one or more of SEQ ID NOs: 1-4, 10-15, 18, 19, 23-25, 27, 29, 31-41, and combinations or cocktails thereof. This includes sequences which can hybridise to such sequences under stringent hybridisation conditions, sequences complementary thereto, sequences containing modified bases, modified backbones, and functional truncations or extensions thereof which possess or modulate pre-RNA processing activity in a PTPN1 gene transcript. More preferably, the antisense oligomer used in the present invention is chosen from the list comprising: SEQ ID NO: 1 or 32-36. Most preferably, the antisense oligomer used in the present invention is SEQ ID NO: 33. Preferably the antisense oligomer leads to exon skipping of exon 2.

The invention extends also to a combination of two or more antisense oligomers capable of binding to a selected target to modify splicing of a PTPN1 gene transcript. The combination may be a cocktail of two or more antisense oligomers, a construct comprising two or more or two or more antisense oligomers joined together for use in an antisense oligomer-based therapy.

The invention provides a method to treat, prevent or ameliorate the effects of a disease associated with PTP1B, comprising the step of:
a) administering to the subject an effective amount of one or more antisense oligomers or pharmaceutical composition comprising one or more antisense oligomers as described herein.

Furthermore, the invention provides a method to treat, prevent or ameliorate a disease associated with PTP1B, comprising the step of:
a) administering to the subject an effective amount of one or more antisense oligomers or pharmaceutical composition comprising one or more antisense oligomers as described herein
   wherein the disease associated with PTP1B is a disease that is: (i) associated with down-regulation of insulin signalling in a subject; (ii) associated with down-regulation of the leptin signal transduction pathway in a subject; (iii) associated with cancer cell growth, migration and invasion. For example, the disease may be T2DM and/or obesity. Alternatively, the disease may be solid cancers.

Preferably, the therapy is used to develop non-functional, truncated or nonsense PTP1B proteins. The decrease in levels of PTP1B is preferably achieved by decreasing the amount of full length transcript level through exon skipping by binding splicing sites and/or modifying pre-mRNA splicing factor binding in the PTPN1 gene transcript or part thereof.

The reduction in PTP1B will preferably lead to a reduction in the quantity, duration or severity of the symptoms of a disease associated with: (i) down-regulation of insulin signalling; (ii) down-regulation of the leptin signal transduction pathway, such as T2DM and/or obesity; and/or (iii) reduction in cancer cell growth, migration and invasion.

According to another aspect of the invention there is provided the use of one or more antisense oligomers as described herein in the manufacture of a medicament for the modulation or control of a disease associated with PTP1B.

The invention also provides for the use of purified and isolated antisense oligomers as described herein, for the manufacture of a medicament for treatment of a disease associated with PTP1B.

There is provided the use of purified and isolated antisense oligomers as described herein for the manufacture of a medicament to treat, prevent or ameliorate the effects of a disease associated with PTP1B.

Preferably, the antisense oligomer used in the present invention is chosen from the list comprising:
SEQ ID NO: 1-4, 10-15, 18, 19, 23-25, 27, 29, 31-41;
SEQ ID NO: 1 or 32-36; or
SEQ ID NO: 33.

Preferably the antisense oligomer leads to exon skipping of exon 2.

The invention extends, according to a still further aspect thereof, to cDNA or cloned copies of the antisense oligomer sequences of the invention, as well as to vectors containing the antisense oligomer sequences of the invention. The invention extends further also to cells containing such sequences and/or vectors.

The invention also provides kits to treat, prevent or ameliorate diseases associated with PTP1B in a subject, which kit comprises at least an isolated or purified antisense oligomer for modifying pre-mRNA splicing factor binding in a PTPN1 gene transcript or part thereof, packaged in a suitable container, together with instructions for its use.

In a preferred embodiment, the kits will contain at least one antisense oligomer as described herein or as shown in Table 1 (SEQ ID NOs: 1-4, 10-15, 18, 19, 23-25, 27, 29, 31-41), or a cocktail of antisense oligomers, as described herein. The kits may also contain peripheral reagents such as buffers, stabilizers, etc.

There is therefore provided a kit to treat, prevent or ameliorate a disease associated with PTP1B in a subject, which kit comprises at least an antisense oligomer described herein or SEQ ID NOs: 1-4, 10-15, 18, 19, 23-25, 27, 29, 31-41 as shown in Table 1 and combinations or cocktails thereof, packaged in a suitable container, together with instructions for its use.

There is also provided a kit to treat, prevent or ameliorate a disease associated with PTP1B in a subject which kit comprises at least an antisense oligomer selected from the group consisting of any one or more of SEQ ID NOs: 1-4, 10-15, 18, 19, 23-25, 27, 29, 31-41, and combinations or cocktails thereof, packaged in a suitable container, together with instructions for its use.

Preferably, the disease associated with PTP1B is a disease that is: (i) associated with down-regulation of insulin signalling in a subject; (ii) associated with down-regulation of the leptin signal transduction pathway in a subject; and/or (iii) associated with cancer cell growth, migration and invasion. For example, the disease may be T2DM and/or obesity. Alternatively, the disease may be solid cancers.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an affected area of the animal, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

Those of ordinary skill in the field should appreciate that applications of the above method have wide application for identifying antisense oligomers suitable for use in the treatment of many other diseases.

The antisense oligomers of the present invention may also be used in conjunction with alternative therapies, such as drug therapies.

The present invention therefore provides a method of treating, preventing or ameliorating the effects of a disease associated with PTP1B, wherein the antisense oligomers of the present invention and administered sequentially or concurrently with another alternative therapy associated with treating, preventing or ameliorating the effects of the disease associated with PTP1B.

If the disease is associated with insulin resistance, T2DM, leptin resistance, or obesity, the alternative therapy may be chosen from the list comprising: insulin and insulin mimetics; agents that increase insulin release (amylin mimetics such as pramlintide; sodium glucose transporter 2 inhibitors such as canagliflozin; incretin mimetics [GLP-1 agonists] such as exenatide or liraglutide; dipeptidyl-peptidase 4 inhibitors such as saxagliptin, sitagliptin or linagliptin; sulfonylureas such as glyburide, glipizide, glimepiride, chlorpropamide, tolazamide, gliquidone, glibenclamide, gliclazide, acetohexamide or tolbutamide; glinides such as nateglinide or repaglinide); agents that decrease absorption of sugar from the intestines (for example acarbose, voglibose, and miglitol); agents that prevent reabsorption of filtered glucose by the kidney (for example dapagliflozin and canagliflozin); agents that make the body more sensitive to insulin (for example metformin, ciglitazone, troglitazone, rosiglitazone and pioglitazone); dietary modifications in conjunction with regular exercise; surgery to increase weight loss.

If the disease is associated with cancer, the alternative therapy may be chosen from the list comprising: chemotherapy, radiation therapy, surgery to excise the solid tumour, immune therapy.

General

Throughout this specification, unless the context requires otherwise, the word “comprise” or variations such as “comprises” or “comprising”, will be understood to imply the inclusion of a stated integer or group of integers but not the exclusion of any other integer or group of integers.

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The invention described herein may include one or more range of values (e.g. concentration). A range of values will be understood to include all values within the range, including the values defining the range, and values adjacent to the range which lead to the same or substantially the same outcome as the values immediately adjacent to that value which defines the boundary to the range.

The following Examples are to be construed as merely illustrative and not limitative of the remainder of the disclosure in any way whatsoever. These Examples are included solely for the purposes of exemplifying the present invention. They should not be understood as a restriction on the broad summary, disclosure or description of the invention as set out above. Without further elaboration, it is believed that one skilled in the art can, using the preceding description, utilize the present invention to its fullest extent. In the foregoing and in the following examples, all temperatures are set forth uncorrected in degrees Celsius; and, unless otherwise indicated, all parts and percentages are by weight.

### EXAMPLES

Further features of the present invention are more fully described in the following description of several non-limiting embodiments thereof. This description is included solely for the purposes of exemplifying the present invention. It should not be understood as a restriction on the broad summary, disclosure or description of the invention as set out above.

### Example 1

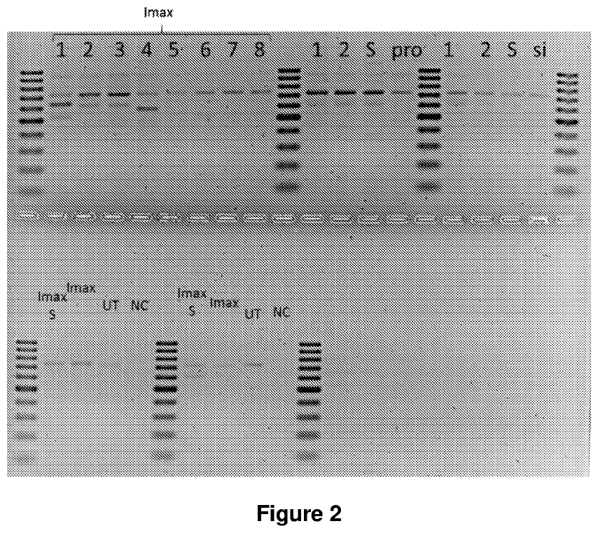

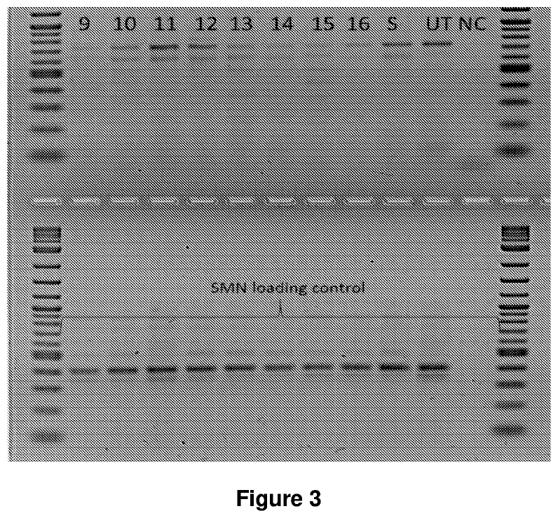

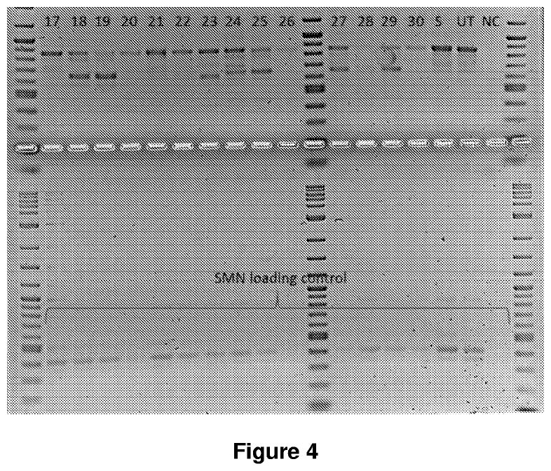

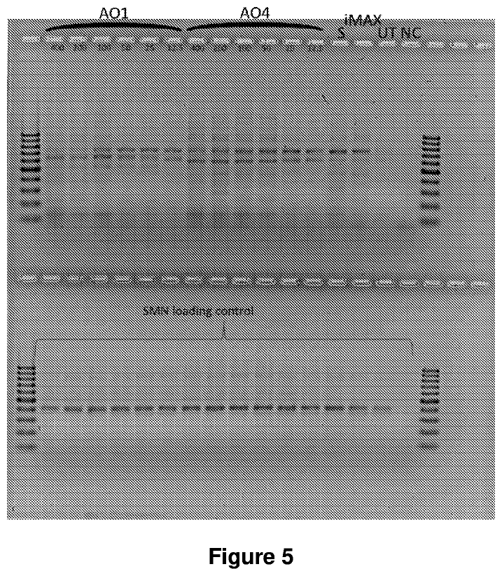

Forty-one ASOs (AOs) targeting human PTPN1 or mouse Ptpn1 transcript were designed and synthesized as shown in Table 1. All these AOs were transfected to Huh-7 and/or HepG2 cell lines (human hepatocarcinoma cell lines); and/or IHH cell line, a normal human liver cell line; and/or AML-12 cell line, a normal mouse liver cell line. Results showed that AO 1-4, 10-15, 18, 19, 23-25, 27, 29, 31-41 can induce exon skipping (FIG. 2-5, 8, 10-15, 17-19).

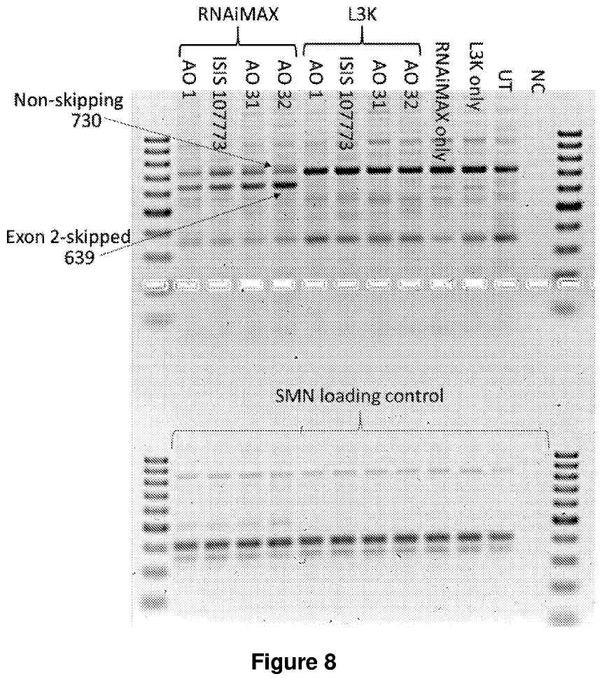

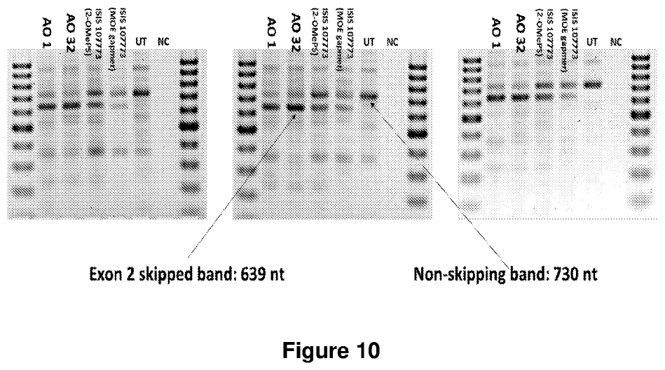

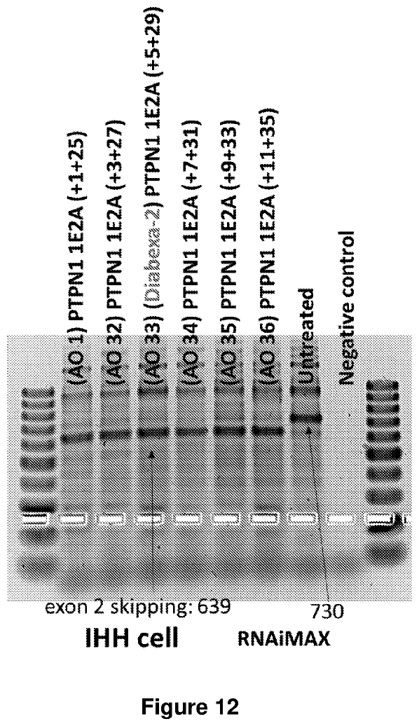

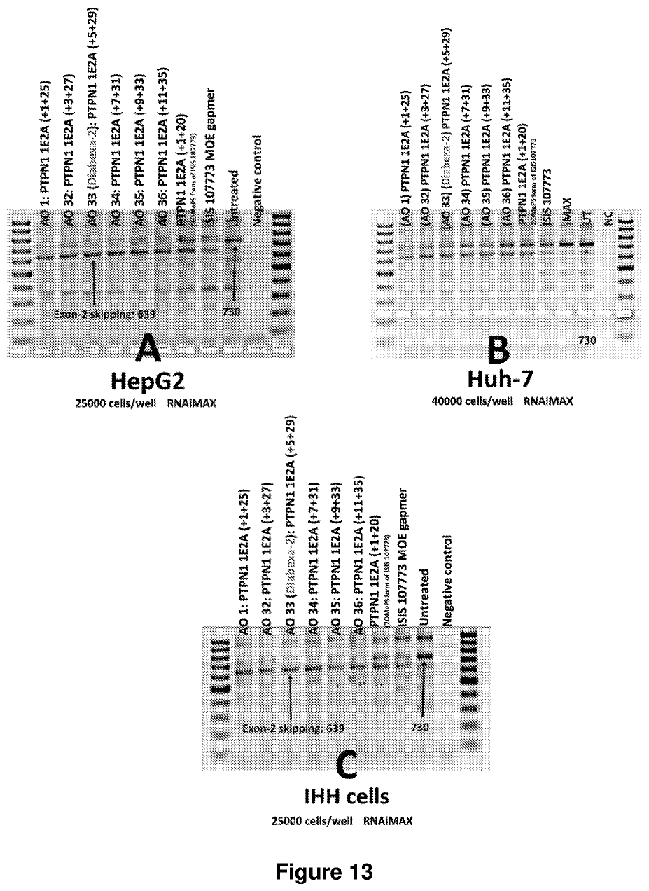

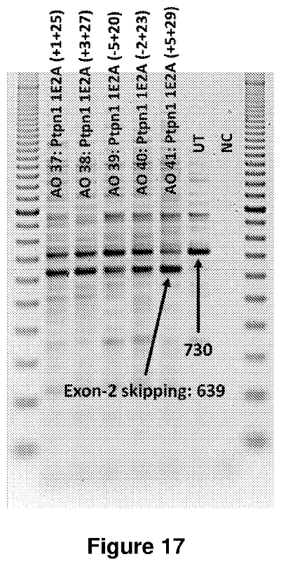

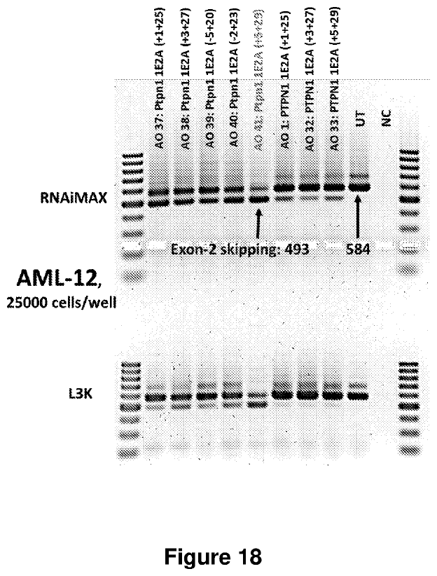

The human PTPN1 exon-2 targeting AOs (AO 1, AO 31-36) showed excellent exon skipping effect on exon-2 when transfected into liver related cell lines such as Huh-7 (FIG. 2, 5, 13B), HepG2 (FIGS. 8, 10, 11, 13A, 14A), IHH (FIG. 12, 13C, 14B, 15), and even mouse AML-12 (FIG. 18, 19). In order to further evaluate the exon-2 skipping effect of AOs in mouse, mouse Ptpn1 exon-2 targeting AOs (AO 37-41) were transfected into both HepG2 (FIG. 17) and AML-12 cells (FIG. 18, 19) and induced efficient exon-2 skipping. AO 1, 32-36 were found to be highly efficient in inducing human PTPN1 exon-2 skipping. Furthermore, AO 33, PTPN1 1E2A (+5+29) (Diabexa-2) showed the best exon-2 skipping efficiency (FIG. 12-14), in addition, its mouse form or version, AO 41, Ptpn1 1E2A (+5+29), also induced the highest percentage of exon-2 skipping in mouse AML-12 cell line (FIG. 18, 19), even in human HepG2 cell line (FIG. 17), in comparison with other mouse Ptpn1 exon-2 targeting AOs (AO 37-40).

General Methods

Design and Synthesis of Antisense Oligonucleotides

Phosphorothioated (PS) 2′-O-methyl (2′OMe) AOs (Table 1) were designed and synthesised in-house on an ABI Expedite® 8909 Nucleic Acid Synthesis System (Applied Biosystems) or GE AKTA Oligopilot 10 synthesizer (GE Healthcare Life Science) via standard phosphoramidite chemistry in 1 μmol scale. The synthesised AOs were deprotected and cleaved from the solid support by treatment with ammonium hydroxide (NH₄OH) at 55° C. for 8 hours. The crude AOs were then desalted by Illustra NAP-10 columns (GE Healthcare). Purified oligonucleotides were then verified by polyacrylamide gel electrophoresis.

Cell Culture and Transfection of ASOs Into Cells

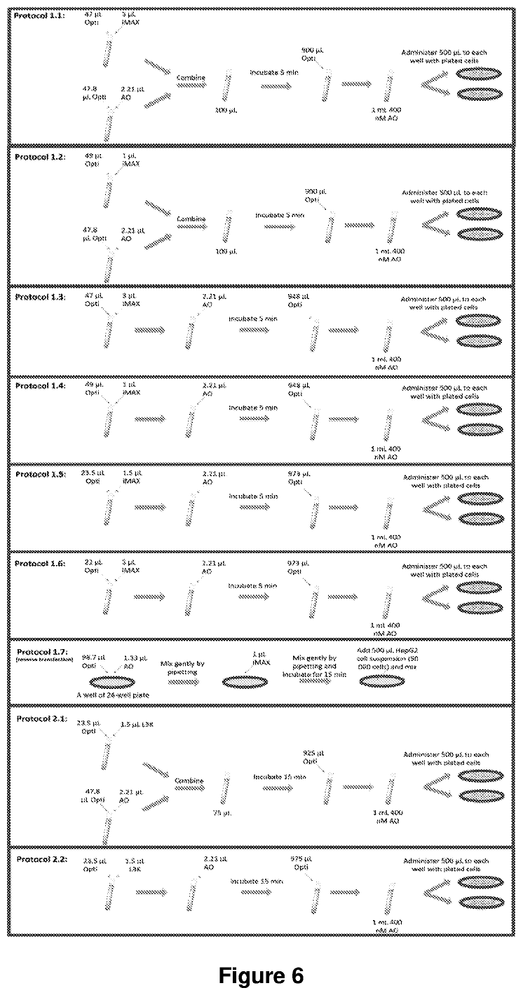

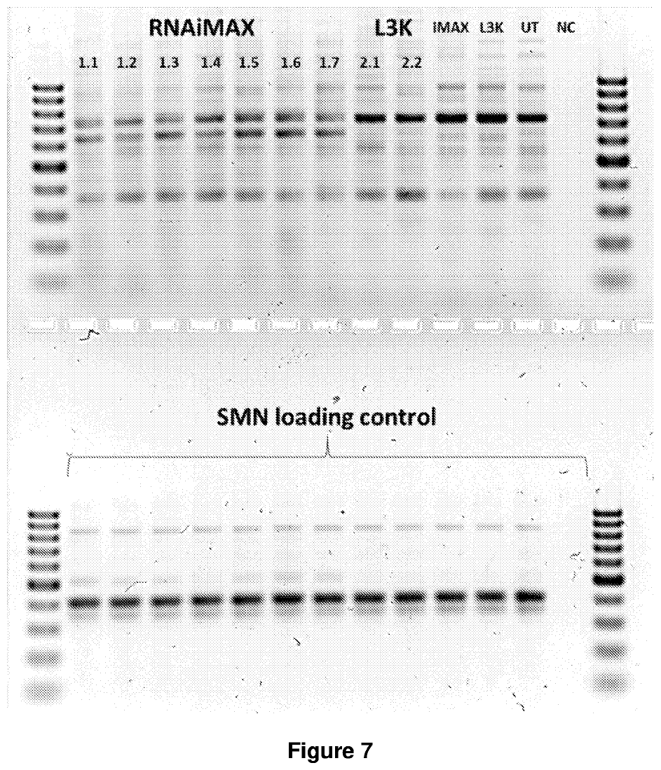

Human liver cancer cell line Huh-7 was obtained from American Type Culture Collection (ATCC); the other human hepatocarcinoma cell line, HepG2 was obtained from European Collection of Authenticated Cell Cultures (ECACC). Huh-7 cell line was cultured in 10% fetal bovine serum (FBS) Dulbecco's Modified Eagle's Medium (DMEM) (Thermo Fisher Scientific), HepG2 cell line was cultured in 10% FBS Eagle's Minimum Essential Medium (ATCC). Both IHH and AML-12 cell lines were cultured in 10% FBS, 1% ITS (Insulin-transferrin-sodium) liquid media supplement (Thermo Fisher Scientific), and 40 ng/mL dexamethasone (Sigma). All of the liver cell lines were cultured in a humidified atmosphere at 55° C., 5% CO₂. Cells were cultured to reach 70-90% confluency, followed by being seeded at a density of 5.0×10⁴ (cells/mL) into 24-well plates (Thermo Fisher Scientific) 24 hours prior to transfection. The day after, the AOs were transfected at 400 nM concentration using RNAiMAX reagent following the original or modified manufacturer's protocols (different modified transfection protocols were based on manufacturer's instructions and shown in FIG. 6) for screening purpose. 24 hours after transfection, the cells were collected for RNA extraction.

RNA Extraction and RT-PCR

RNA was extracted from transfected cells using Direct-zol™ RNA MinPrep Plus with TRI reagent (Zymo Research) as per the manufacturer's instructions. The human PTPN1 exon-2 skipping products (product size: 639 bp) and non-skipping products (product size: 730 bp), and mouse Ptpn1 exon-2 skipping products (product size: 493 bp) and non-skipping products (product size: 584 bp), were amplified using SuperScript® III one-step RT-PCR kit (Thermo Fisher Scientific) with human PTPN1 primer pair (PTP1B_Ex1F: 5′-GTG ATG CGT AGT TCC GGC TG-3′ (SEQ ID NO: 42); PTP1B_Ex6R: 5′-CAG GGA CTC CAA AGT CAG GC-3′ (SEQ ID NO″ 43)) or mouse Ptpn1 primer pair (Ptpn1_B_Ex1F: 5′-AGA TCG ACA AGG CTG GGA AC-3′ (SEQ ID NO:44); Ptpn1_B_Ex6R: 5′-TGA GCC TGA CTC TCG GAC TT-3′ (SEQ ID NO:45)). Briefly, the conditions were 55° C., 30 minutes; 94° C., 2 minutes followed by 33 cycles of 94° C., 30 seconds, 60° C., 1 minute and 68° C., 2 minutes. The PCR products were then separated on a 2% agarose gel in Tris-acetate-EDTA buffer and visualized with Fusion Fx gel documentation system. Densitometry was performed by ImageJ software.

Sequencing

Bandstab technique was performed following the guidelines from Anthony and James (1992). The bandstab samples were then amplified with the same primer set mentioned above using AmpliTaq Gold® DNA Polymerase kit (Thermo Fisher Scientific). Briefly, the conditions were 94° C., 6 minutes followed by 32 cycles of 94° C., 30 seconds, 55° C., 1 minute and 72° C., 2 minutes. PCR products were confirmed by 2% agarose gel and sent to AGRF (Australian Genome Research Facility) for Sanger sequencing using both the forward primer and reverse primer mentioned above.

### Example 2

The sequence of PTPN1 1E2A (+1+25) (AO1) and PTPN1 1E2A (+3+27) (AO 32) are similar to one of the AOs already patented by Ionis Pharmaceuticals: PTPN1 1E2A (+1+20) (ISIS 107773). AO 1 and AO 32 are 2′-OMePS chemistry, while ISIS 107773's chemistry is 5-10-5 MOE (2′-O-methoxyethyl) gapmer. Comparison between 2′OMePS form of AO 1, 32-36, 2′OMePS form of ISIS 107773, and 5-10-5 MOE gapmer form of ISIS 107773 was made in their ability in inducing PTPN1 exon-2 skipping (FIG. 8, 10, 13, Table 2). Data showed that AO 33 (Diabexa-2) is the best performing AO in inducing PTPN1 exon-2 skipping and/or full length transcript knockdown. Moreover, the sequence similarity of AO 33-36 to ISIS 107773 is less than 70% (Table 3).

TABLE 2
Comparison of PTPN1 exon-2 skipping efficacy and full length transcript
knockdown efficacy between AO 1, 32-36, and ISIS 107773.

| Huh-7 | | | HepG2 | | | IHH | | |
|---|---|---|---|---|---|---|---|---|
| Exon-2 skipping efficacy | | Full length | Exon-2 skipping efficacy | | Full length | Exon-2 skipping efficacy | | Full length |
| Full | Exon-2 | knockdown | Full | Exon-5 | knockdown | Full | Exon-2 | knockdown |
| length | skipped | efficacy | length | skipped | efficacy | length | skipped | efficacy |

AO 1: PTPN1 1E2A (+1 + 25)

| | | | | | | | | |
|---|---|---|---|---|---|---|---|---|
| 26% | 74% | 74% | 7% | 93% | 93% | 9% | 91% | 91% |

AO 32: PTPN1 1E2A (+3 + 27)

| | | | | | | | | |
|---|---|---|---|---|---|---|---|---|
| 29% | 71% | 71% | 18% | 82% | 82% | 14% | 86% | 86% |

AO 33 (Diabexa-2): PTPN1 1E2A (+5 + 29)

| | | | | | | | | |
|---|---|---|---|---|---|---|---|---|
| 23% | 77% | 77% | 5% | 95% | 95% | 0% | 100% | 100% |

AO 33: PTPN1 1E2A (+7 + 31)

| | | | | | | | | |
|---|---|---|---|---|---|---|---|---|
| 38% | 62% | 62% | 6% | 94% | 94% | 0% | 100% | 100% |

AO 34: PTPN1 1E2A (+9 + 33)

| | | | | | | | | |
|---|---|---|---|---|---|---|---|---|
| 27% | 73% | 73% | 11% | 89% | 89% | 0% | 100% | 100% |

AO 36: PTPN1 1E2A (+11 + 35)

| | | | | | | | | |
|---|---|---|---|---|---|---|---|---|
| 43% | 57% | 57% | 11% | 89% | 89% | 0% | 100% | 100% |

ISIS 107773: PTPN1 1E2A (+1 + 20) (2′OMePS)

| | | | | | | | | |
|---|---|---|---|---|---|---|---|---|
| 45% | 55% | 55% | 29% | 71% | 71% | 37% | 63% | 63% |

ISIS 107773: PTPN1 1E2A (+1 + 20) (5-10-5 MOE gapmer)

| | | | | | | | | |
|---|---|---|---|---|---|---|---|---|
| 66% | 34% | 50% | 16% | 84% | 88% | 26% | 74% | 80% |

TABLE 3
Sequence comparison between AO 1, 32-36, and ISIS 10773

| ISIS107773 | CTG GCT TCA TGT CGG ATA TC | Similarity | |
|---|---|---|---|
| AO 1: PTPN1 1E2A (+1+25) | AGU CAC UGG CUU CAU GUC GGA UAU C | 80% | SEQ ID NO: 46 |
| AO 32: PTPN1 1E2A (+3+27) | GAA GUC ACU GGC UUC AUG UCG GAU A | 72% | SEQ ID NO: 1 |
| AO 33 (Dilbexa-2): | GGG AAG UCA CUG GCJ UCA UGU CGG A | 64% | SEQ ID NO: 32 |
| PTPN1 1E2A (+5+29) | | | |
| AO 34: PTPN1 1E2A (+7+31) | AUG GGA AGU CAC UGG CUU CAU GUC G | 56% | SEQ ID NO: 33 |
| AO 33: PTPN1 1E2A (+9+33) | ACA UGG GAA GUC ACU GGC UUC AUG U | 48% | SEQ ID NO: 34 |
| AO 351 PTPN1 1E2A (+11+35) | CUA CAU GGG AAG UCA CUG GCU UCA U | 40% | SEQ ID NO: 35 |

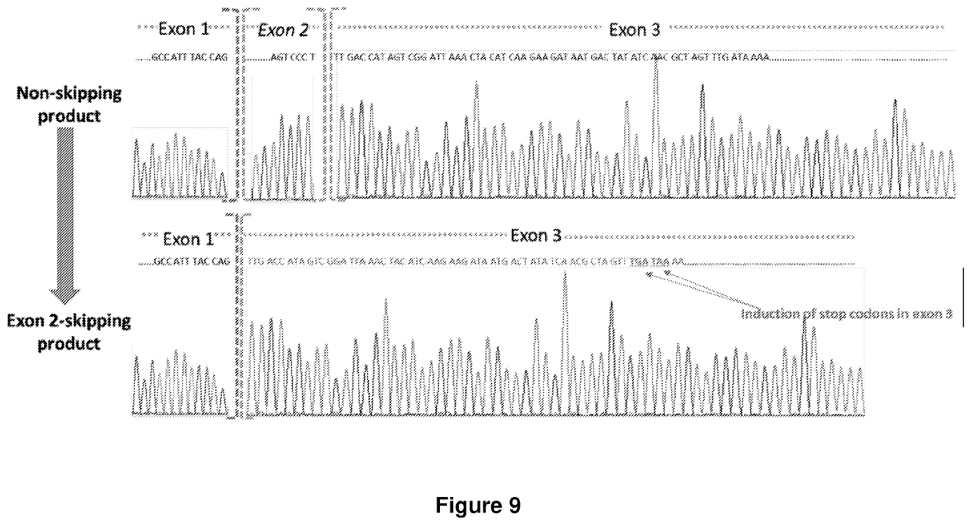

The PTPN1 exon-2 targeting AOs (AO 1, AO 31-36) ability in inducing exon-2 skipping has been confirmed. For example, AO 1's ability to induce exon-2 skipping has been confirmed by Sanger Sequencing (FIG. 9).

### Example 3

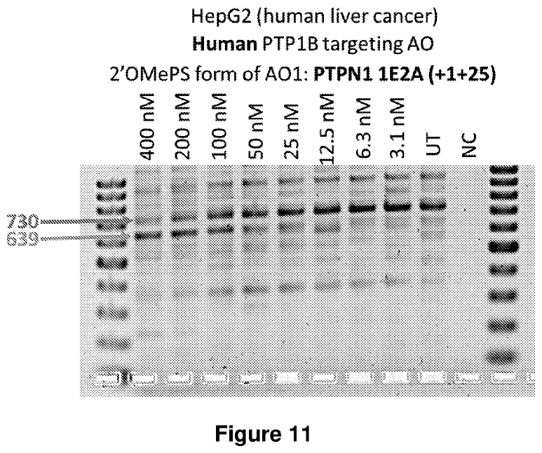

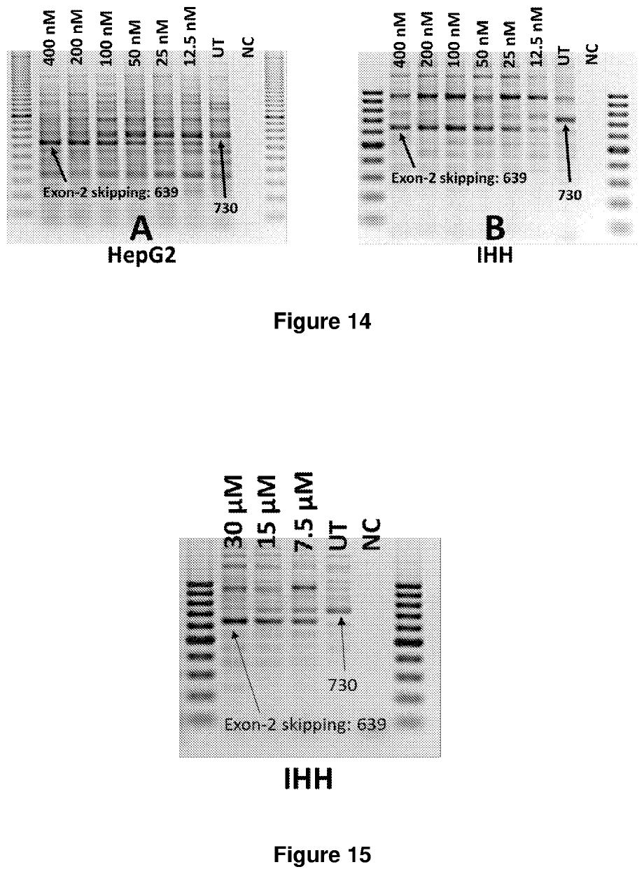

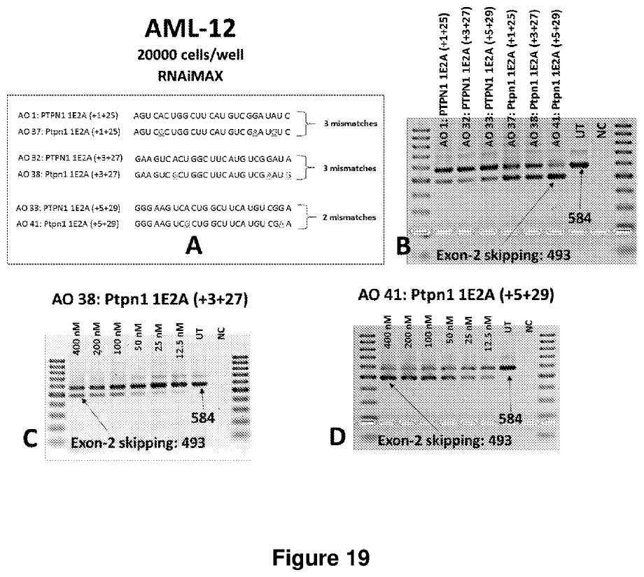

Different concentrations (400, 200, 100, 50, 25, 12.5 nanomolar) of human PTPN1 or mouse Ptpn1 exon-2 targeting AOs were transfected into different types of liver related cells and showed dose dependency. For example, AO 1 induced efficient exon-2 skipping in HepG2 cells in a dose dependence manner (FIG. 11), AO 33 (Diabexa-2) induced efficient exon-2 skipping in both HepG2 and IHH cells in a dose dependence manner (FIG. 14), AO 38 (mouse version of AO 32) and AO 41 (mouse version of AO 33) induced efficient exon-2 skipping in mouse AML-12 cells in a dose dependence manner (FIG. 19C, D). All these results described above confirmed that the PTPN1 exon-2 targeting AOs (AO 1, AO 32-36), most preferably, AO 33 (Diabexa-2), PTPN1 1E2A (+5+29) induce significant exon-2 skipping of PTPN1 transcript thus these AOs may potentially induce reduction of functional PTP1B protein production.

Results clearly showed that AO 33 (Diabexa-s), PTPN1 1 E2A (+5+29) has better human PTPN1 exon-2 skipping efficiency and non-skipping product knockdown efficiency than other exon-2 targeting AOs including AO 1, 32, 34-36 and both 2′-OMePS and 5-10-5 MOE gapmer form of ISIS 107773 (FIG. 12-14). Furthermore, the mouse form of AO 33 (Diabexa-2), i.e., AO 41, showed the best mouse Ptpn1 exon-2 skipping effect compared to other Ptpn1 exon-2 targeting AOs (AO 37-40) (FIG. 17-19).

### Example 4

Phosphorodiamidate Morpholino Oligomer (PMO) form of exon-2 targeting AOs were synthesized and evaluated. For example, PMO form of AO 33 (Diabexa-2) was transfected into IHH cells via nucleofection and showed efficient PTPN1 exon-2 skipping in a dose dependence manner (FIG. 15). All the results described above confirmed that the AO 33 (Diabexa-2) induces significant exon-2 skipping of PTPN1 transcript thus this AO may potentially induce reduction of functional PTP1B protein production.

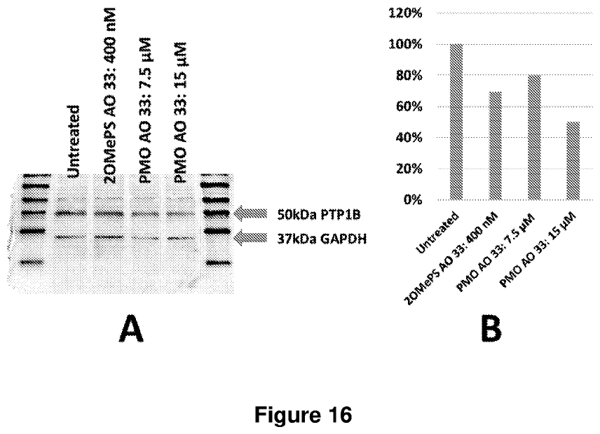

Western blotting was performed to evaluate the effect of 2′OMePS form and PMO form of PTPN1 1E2A (+5+29) towards inhibition of PTP1B protein in comparison to untreated sample. 72 hours after transfection, IHH cells were harvested and stored in −80° C. freezer. Frozen transfected IHH cell pellets were thawed and homogenized in SDS lysis buffer (0.5 M Tris-HCL pH 6.8, 3% SDS (w/v) and 10% glycerol (v/v)) containing protease inhibitor (Sigma). The homogenate was then centrifuged at 14 000 g for 3 minutes, after which the supernatant was removed, and the protein concentration of supernatant was estimated using Pierce™ BCA Protein Assay kit (Thermo Fisher Scientific). Protein from the samples was then resolved on a nitrocellulose membrane (Biorad). The membrane was incubated with primary anti-PTP1B antibody (1:1000) (Cat. 5311S, Cell Signaling Technology) in 5% skim milk in TBS-T overnight at 4° C. on see-saw shaker. The membrane was then washed by TBS-T on a shaker at room temperature for 1 hour. After washing, the membrane was incubated with secondary anti-rabbit HRP antibody (1:10000) (Cat. 31460, Thermo Fisher Scientific) on see-saw shaker at room temperature for 1 hour, followed by TBS-T based washing. The protein bands were visualized with a chemiluminescence-based procedure using the Clarity Western ECL detection kit according to the manufacturer's instructions (Biorad). Western blotting results showed that PTPN1 1 E2A (+5+29) (AO 33) treatment significantly reduced the expression level of PTP1B protein (FIG. 16). Specifically, 400 nanomolar of 2′OMePS form of AO 33 (Diabexa-2) induced 31% of PTP1B protein reduction or inhibition, 7.5 μM and 15 μM of PMO form of AO 33 (Diabexa-2) induced 20% and 50% of PTP1B protein reduction or inhibition (FIG. 16).

AO 1, 31-36 showed excellent human PTPN1 exon-2 skipping, resulting in the induction a pre-mature stop codon in exon-3, and leads to significant reduction of the expression level of functional PTPN1 gene product. Furthermore, Western blotting result have proved that AO 33 (Diabexa-2): PTPN1 1E2A (+5+29) treatment significantly reduced the expression level of PTP1B protein.

### Example 5

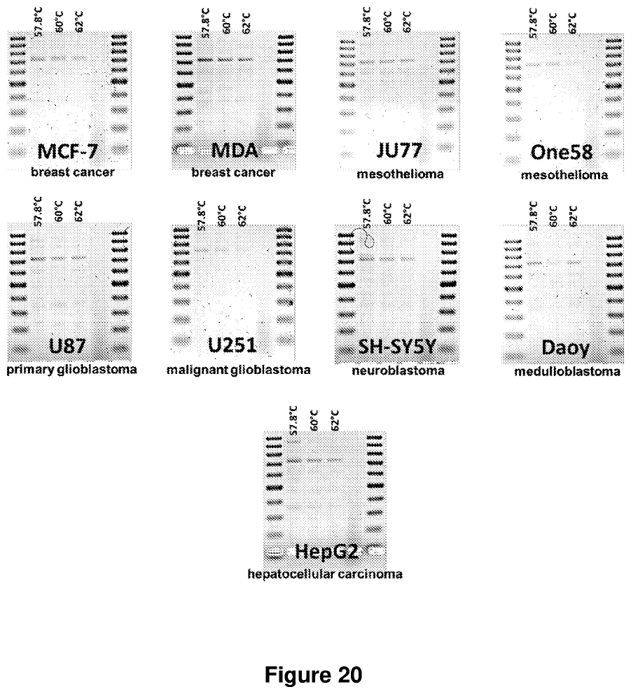

PTP1B expression was analysed in various cancer cells including breast cancer (MCF-7, MDA), mesothelioma (JU77, One58), glioblastoma (U87, U251), neuroblastoma (SH-SY5Y), medulloblastoma (DAOY), and liver cancer (HepG2), which showed a high expression of PTP1B (FIG. 20) using primer pair that gives the product of 730 bp (PTP1B_Ex1F: 5′-GTG ATG CGT AGT TCC GGC TG-3′ (SEQ ID NO: 42); PTP1B_Ex6R: 5′-CAG GGA CTC CAA AGT CAG GC-3′ (SEQ ID NO: 43)). Briefly, the conditions were 55° C., 30 min; 94° C., 2 min followed by 30 cycles of 94° C., 30 sec, 60° C., 1 min and 68° C., 2 min. The PCR products were then separated on a 2% agarose gel in Tris-acetate-EDTA buffer and visualized with Fusion Fx gel documentation system.

1. An isolated or purified antisense oligomer targeted to a nucleic acid molecule encoding PTPN1 pre-mRNA, wherein the antisense oligomer has a nucleobase sequence selected from the list comprising SEQ ID NO: 1-4, 10-15, 18, 19, 23-25, 27, 29, 31-41 which has a modified backbone structure and sequences with at least 80% sequence identity to SEQ ID NO: 1-4, 10-15, 18, 19, 23-25, 27, 29, 31-41 which have a modified backbone structure, and wherein the antisense oligomer inhibits the expression of PTP1B.

2. The antisense oligomer of claim 1 that induces alternative splicing of PTPN1 pre-mRNA through exon skipping.

3. The antisense oligomer of claim 1 that induces exon skipping of exon 2.

4. The antisense oligomer of claim 1 wherein the antisense oligomer contains one or more nucleotide positions subject to an alternative chemistry or modification chosen from the list comprising: (i) modified sugar moieties; (ii) resistance to RNase H; (iii) oligomeric mimetic chemistry.

5. The antisense oligomer of claim 1 wherein:
a) the antisense oligomer is further modified by: (i) chemical conjugation to a moiety; and/or (ii) tagging with a cell penetrating peptide; and/or
b) if a uracil is present in the antisense oligomer, the uracil (U) of the antisense oligomer is replaced by a thymine (T)

6. (canceled)

7. The antisense oligomer of claim 1 that is:
i) a phosphoridoiamidate morpholino oligomer (PMO), 2′-O-Methyl RNA oligomer (2′-OMe) or 2′-O-Methoxyethyl RNA (2′-O-MOE); and/or
ii) SEQ ID NO: 33

8.-9. (canceled)

10. A method for inducing alternative splicing of PTPN1 pre-mRNA, the method including the step of:
providing one or more of the antisense oligomers according to claim 1 and allowing the oligomer(s) to bind to a target nucleic acid site.

11. A pharmaceutical, prophylactic, or therapeutic composition to treat, prevent or ameliorate the effects of a disease associated with PTP1B in a subject, the composition comprising:
one or more antisense oligomers according to claim 1; and
one or more pharmaceutically acceptable carriers and/or diluents.

12. The pharmaceutical composition of claim 11 wherein the disease associated with PTP1B is type 2 diabetes, obesity, and solid cancers.

13. A method of treating, preventing or ameliorating the effects of a disease associated with PTP1B, the method comprising the step of:
administering to the subject an effective amount of one or more antisense oligomers or pharmaceutical composition comprising one or more antisense oligomer according to claim 1.

14. The method of treatment of claim 13 wherein the disease associated with PTP1B is type 2 diabetes, obesity and solid cancers.

15. An expression vector comprising the antisense oligomer according to claim 1.

16.-18. (canceled)

19. A kit to treat, prevent or ameliorate the effects of a disease associated with PTP1B in a subject, which kit comprises at least an antisense oligomer according to claim 1, packaged in a suitable container, together with instructions for its use.

20. The kit of claim 19 wherein the disease associated with PTP1B is type 2 diabetes, obesity, and solid cancers.


SEQUENCE LISTING (46 sequences)

SEQ ID NO: 1 - DNA, 25 bp, Homo sapiens
agucacuggcuucaugucggauauc

SEQ ID NO: 2 - DNA, 25 bp, Homo sapiens
uagucauuaucuucuugauguaguu

SEQ ID NO: 3 - DNA, 25 bp, Homo sapiens
uguaacuccuuugggcuucuuccau

SEQ ID NO: 4 - DNA, 25 bp, Homo sapiens
augacuugauaucuucagagaucaa

SEQ ID NO: 5 - DNA, 25 bp, Homo sapiens
gggaaagcuccuuccacugauccug

SEQ ID NO: 6 - DNA, 25 bp, Homo sapiens
cauuguguggcuccaggauucguuu

SEQ ID NO: 7 - DNA, 25 bp, Homo sapiens
ucucuuccuucacccacuggugauu

SEQ ID NO: 8 - DNA, 25 bp, Homo sapiens
cgaugccguagggugcggcauuuaa

SEQ ID NO: 9 - DNA, 25 bp, Homo sapiens
gcuccggggcgcucccgcaccuggu

SEQ ID NO: 10 - DNA, 25 bp, Homo sapiens
auauccuacaaaaaagaauaaagac

SEQ ID NO: 11 - DNA, 25 bp, Homo sapiens
ccggccacguggauacuuacaggga

SEQ ID NO: 12 - DNA, 25 bp, Homo sapiens
gucaacugaaagacaaaccagaacu

SEQ ID NO: 13 - DNA, 25 bp, Homo sapiens
aauucagacaaucugcuuaccuggg

SEQ ID NO: 14 - DNA, 25 bp, Homo sapiens
gggcccugcaaagacacaauaacac

SEQ ID NO: 15 - DNA, 25 bp, Homo sapiens
caaaugaagccgagacuuaccgaac

SEQ ID NO: 16 - DNA, 25 bp, Homo sapiens
uuuaacuggggaaacaaauaauagu

SEQ ID NO: 17 - DNA, 25 bp, Homo sapiens
gaagugugugcuauacucacuguaa

SEQ ID NO: 18 - DNA, 25 bp, Homo sapiens
ugggucugaaagagaaaaauacuca

SEQ ID NO: 19 - DNA, 25 bp, Homo sapiens
acccgcgagggccuccuuaccagca

SEQ ID NO: 20 - DNA, 25 bp, Homo sapiens
uccaucugaaagccagagaggagau

SEQ ID NO: 21 - DNA, 25 bp, Homo sapiens
caaacaaaggcaaugcugaccugca

SEQ ID NO: 22 - DNA, 25 bp, Homo sapiens
ugauccuugaaagagcagcaagagg

SEQ ID NO: 23 - DNA, 25 bp, Homo sapiens
cugggacccaaucauauuaccuuuc

SEQ ID NO: 24 - DNA, 25 bp, Homo sapiens
ucaugcugaggaaucagagggcaga

SEQ ID NO: 25 - DNA, 25 bp, Homo sapiens
gucugucaguggaaacauacccugu

SEQ ID NO: 26 - DNA, 25 bp, Homo sapiens
aggaacuggaaugaaaccaaacagu

SEQ ID NO: 27 - DNA, 25 bp, Homo sapiens
cgacuucuaacuucagugucuugac

SEQ ID NO: 28 - DNA, 25 bp, Homo sapiens
ugacggcuccccuuuggcuggggag

SEQ ID NO: 29 - DNA, 25 bp, Homo sapiens
ucagugcaugguccucguccuucuc

SEQ ID NO: 30 - DNA, 25 bp, Homo sapiens
agagguaagcgccggccgugaggac

SEQ ID NO: 31 - DNA, 23 bp, Homo sapiens
ucacuggcuucaugucggauauc

SEQ ID NO: 32 - DNA, 25 bp, Homo sapiens
gaagucacuggcuucaugucggaua

SEQ ID NO: 33 - DNA, 25 bp, Homo sapiens
gggaagucacuggcuucaugucgga

SEQ ID NO: 34 - DNA, 25 bp, Homo sapiens
augggaagucacuggcuucaugucg

SEQ ID NO: 35 - DNA, 25 bp, Homo sapiens
acaugggaagucacuggcuucaugu

SEQ ID NO: 36 - DNA, 25 bp, Homo sapiens
cuacaugggaagucacuggcuucau

SEQ ID NO: 37 - DNA, 25 bp, Homo sapiens
agucgcuggcuucaugucgaauguc

SEQ ID NO: 38 - DNA, 25 bp, Homo sapiens
gaagucgcuggcuucaugucgaaug

SEQ ID NO: 39 - DNA, 25 bp, Homo sapiens
cuggcuucaugucgaauguccuaca

SEQ ID NO: 40 - DNA, 25 bp, Homo sapiens
ucgcuggcuucaugucgaauauccu

SEQ ID NO: 41 - DNA, 25 bp, Homo sapiens
gggaagucgcuggcuucaugucgaa

SEQ ID NO: 42 - DNA, 20 bp, Homo sapiens
gtgatgcgtagttccggctg

SEQ ID NO: 43 - DNA, 20 bp, Homo sapiens
cagggactccaaagtcaggc

SEQ ID NO: 44 - DNA, 20 bp, Mus musculus
agatcgacaaggctgggaac

SEQ ID NO: 45 - DNA, 20 bp, Mus musculus
tgagcctgactctcggactt

SEQ ID NO: 46 - DNA, 20 bp, Homo sapiens
ctggcttcatgtcggatatc